(19) United States
(12) Patent Application Publication
(10) Pub. No.: US 2014/0018251 A1
(43) Pub. Date: Jan. 16, 2014

(54) Methods for Predicting Response to Anti-Cancer Therapy in Cancer Patients

(71) Applicants: Sabine Charlotte Linn, Amsterdam (NL); Marieke Anne Vollebergh, Amsterdam (NL); Petra Marleen Nederlof, Amsterdam (NL)

(72) Inventors: Sabine Charlotte Linn, Amsterdam (NL); Marieke Anne Vollebergh, Amsterdam (NL); Petra Marleen Nederlof, Amsterdam (NL)

(73) Assignee: Stichting Het Nederlands Kanker Instituut, Amsterdam (NL)

(21) Appl. No.: 13/825,286

(22) PCT Filed: Sep. 20, 2011
(86) PCT No.: PCT/IB11/02759

§ 371 Date: Oct. 4, 2013

Related U.S. Application Data
(60) Provisional application No. 61/384,499, filed on Sep. 20, 2010.

(51) Int. Cl. C12Q 1/68

(52) U.S. Cl. CPC C12Q 1/6886
USPC 506/9; 435/6.11; 506/16

(57) ABSTRACT

Methods for optimizing the therapeutic efficacy of anti-cancer therapy by detecting phenotypic genetic traits using comparative genomic hybridization are disclosed.

### CROSS-REFERENCE TO RELATED APPLICATIONS

This PCT application claims priority to U.S. provisional patent application No. 61/384,499, filed Sep. 20, 2010 and entitled, Methods for Predicting Response to Anti-Cancer Therapy in Cancer Patients, the contents of which are incorporated herein by reference, in their entirety.

### FIELD

Methods provided by the present disclosure relate to optimizing the therapeutic efficacy of anti-cancer therapy by detecting phenotypic genetic traits using comparative genomic hybridization.

### BACKGROUND

Breast cancer is the most frequently occurring cancer among women in the western world. It is a heterogeneous cancer disease, consisting of several subtypes.

Molecular biology has greatly enhanced our understanding of the heterogeneity of breast cancer, but few molecular tumor features are actually used in the clinic to guide the choice of a systemic treatment strategy.

Neoadjuvant systemic therapy, or administration of therapeutic agents prior to a main treatment, has become a widely used treatment strategy for patients with early, or locally advanced, breast cancer. Despite its early and late toxicities, this treatment strategy reduces the risk of breast cancer relapse and mortality by approximately half.

In spite of these advantages, a disadvantage to the use of neoadjuvant systemic therapy is the lack of predictive tests to individualize the choice of certain combinations of drugs for an individual breast cancer patient to ensure maximal benefit with minimal toxicity. For example, for highly toxic adjuvant treatment regimens, such as high dose alkylating chemotherapy with hematopoietic stem-cell rescue, the survival benefit when compared with standard chemotherapy increases by approximately 10% for patients with 10 or more positive axillary lymph nodes. It would thus be advantageous to be able to target those 10% of patients who would benefit from high dose alkylating chemotherapy. However, no such predictive test presently exists. Because of the relatively high toxicity and the low level of efficacy in unselected breast cancer patients, alkylating agents are not commonly used in the treatment of breast cancer, with the exception of cyclophosphamide.

Alkylating chemotherapy and platinating agents work by causing interstrand DNA crosslinking, which cause DNA double strand breaks. In normal cells, these double strand breaks are repaired by a process called homologous recombination. If this process is unavailable or impaired, a situation referred to as “homologous recombination deficiency” exists and alternative, error-prone DNA repair mechanisms take over, leading to genomic instability. The breast cancer genes BRCA1 and BRCA2 are involved in normal homologous recombination and tumors of patients carrying germ-line inactivating mutations in one or both of these genes show homologous recombination deficiency. BRCA1 and BRCA2 can also be inactivated in sporadic cancers as well, a phenomenon sometimes referred to as BRCA-likeness. Emerging preclinical evidence shows that breast cancers with a defective DNA repair system, such as a mutation in the BRCA1 or BRCA2 genes, may be extremely sensitive to DNA damaging agents, such as platinum compounds and bifunctional alkylating agents. It therefore appears that patients with breast cancers harboring a defective DNA repair system may specifically benefit from high dose alkylating chemotherapy, an intensive DNA double strand break (DSB)-inducing regimen.

Tumors with homologous recombination deficiency have been shown to be particularly sensitive to DNA crosslinking agents, such as alkylators and platinum drugs or platinating agents. Both classes of drugs are employed in advanced breast cancer. The novel poly(ADP-ribose) polymerase inhibitors (PARP inhibitors) are specifically effective in homologous recombination deficient tumors as well, and have shown impressive activity in clinical studies recently. Unfortunately, no clinical tests exist which can reliably determine homologous recombination deficiency in tumor biopsies.

### SUMMARY

Therefore, methods of optimizing the therapeutic efficacy of anti-cancer therapies by identifying patients who would benefit from one or more anti-cancer therapies, including, without limitation, DNA double strand break-inducing regimens such as high dose alkylating chemotherapy, by reliably determining homologous recombination deficiency in tumor biopsies, and by identifying patients with breast cancers harboring a defective DNA repair system, are useful. In various aspects, the DNA double strand break-inducing regimens can be intensive direct DNA double strand break-inducing regimens, intensive indirect DNA double strand break-inducing regimens, moderate direct DNA double strand break-inducing regimens, moderate indirect DNA double strand break-inducing regimens, weak direct DNA double strand break-inducing regimens, weak indirect DNA double strand break-inducing regimens, and/or combinations thereof.

The present disclosure is based on the discovery that certain chromosomal copy number aberrations in tumor cells allow tumors to be classified as either BRCA1-associated tumors, or sporadic tumors. The classification of a tumor in this manner allows for the prospective prediction of responsiveness of the patient from which the tumor was removed to anti-cancer therapy.

In a first aspect, methods for using a BRCA1 aCGH classifier to detect genomic copy number variations in a test sample, as compared to a reference sample, in the genomic loci 1p35-21, 3q22-27, 5p13, 5q21-34, 6p25-22, 7p21-15, 7q31-36, 8q22-24, 10p15-14, 10p12, 12p13, 12q21-23, 13q31-33, 14q22-24, 15q14-21 and 21q11-22 are disclosed. The methods comprise detecting genomic copy number variations in a test sample in at least one, or a plurality, of the genomic loci selected from 1p35-21, 3q22-27, 5p13, 5q21-34, 6p25-22, 7p21-15, 7q31-36, 8q22-24, 10p15-14, 10p12, 12p13, 12q21-23, 13q31-33, 14q22-24, 15q14-21 and 21q11-22, wherein a variation in copy number at any one or more of the genomic loci, as compared to the number of copies per cell of DNA from a reference sample, classifies the cell sample as from a BRCA1-associated tumor, and wherein such classification can be used to predict an individual subject's response to anti-cancer therapy. In some embodiments, the genomic copy number variations are detected at all 16 genomic loci. In some embodiments, the genomic copy number variations are detected at a number of genomic loci selected from greater than 1, greater than 2, greater than 3, greater than 4, greater than 5, greater than 6, greater than 7, greater than 8, greater than 9, greater than 10, greater than 11, greater than 12, greater than 13, greater than 14 and greater than 15. In some embodiments, the genomic copy number variations are detected at a number of genomic loci selected from less than 16, less than 15, less than 14, less than 13, less than 12, less than 11, less than 10, less than 9, less than 8, less than 7, less than 6, less than 5, less than 4, less than 3, and less than 2.

### BRIEF DESCRIPTION OF THE DRAWINGS

Those skilled in the art will understand that the drawings, described herein, are for illustration purposes only. The drawings are not intended to limit the scope of the present disclosure.

FIG. 1 depicts BRCA1-associated genomic loci used to identify breast cancers with homologous recombination deficiency due to a defect in the BRCA1 pathway.

FIG. 2 depicts exemplary BAC clones that may be used to detect, or to generate probes to detect, copy number aberrations in the genomic loci of FIG. 1.

FIG. 3 depicts relevant patient data and the protocols used for array comparative genomic hybridization in Example 1.

FIG. 4 depicts the mutation analysis for Example 1; the investigators screened for the most common mutations reported in Dutch families known to carry pathogenic germline BRCA1 or BRCA2 mutations.

FIG. 5 is a flow diagram of patients from the MBC-series of Example 1. Flow of patients through the study, including number of patients in each stage, is depicted. Reasons for dropout are listed. *=These two patients did not confer to the selection criteria and were classified as stage IIIc according to American Joint Committee on Cancer (AJCC) Staging Manual 2002. †=This patient did not confer to the selection criteria: she had a ductal carcinoma in the right breast with one positive lymph node (ER−, PR+) for which she had a mastectomy followed by 6 cycles of CMF. Three years later ductal carcinoma in her left breast was detected and she had a lumpectomy (diameter 0.9 cm), lymph node dissection and radiotherapy. Eight years later she had a recurrence of the ductal carcinoma in her left breast for which she had a mastectomy (diameter 2 cm, irradical resection, ER−, PR−). Ten months later a metastasis in the left adrenal gland was discovered which was surgically extracted (ER−,PR−). Three months later lung, liver, bone and soft tissue metastases developed for which she was treated with bifunctional alkylating chemotherapy. Review of the histology showed morphologic resemblance and an identical cell type of the adrenal gland metastasis and the most recent tumor in the left breast. DNA was extracted from the most recent breast cancer tumor of the left breast. Abbreviations: FEC=5-fluorouracil, epirubicin, cyclophosphamide.

FIG. 6 depicts the univariate Cox proportional-hazard regression analysis of the risk of tumor progression after HD chemotherapy in MBC series patients with a univariate HR for progression of 0.31 (95% CI: 0.14-0.66).

FIG. 7 depicts the univariate Cox proportional-hazard regression analysis of the risk of tumor progression after HD chemotherapy in MBC series patients, wherein adjustment for potential confounders did not substantially modify the HR.

FIG. 8 depicts the types of mutations found to be present in the MBC series patients.

FIG. 9 is a flow diagram of patients from the stage-III series. Flow of patients through the study including number of patients in each stage. Reasons for dropout are listed. Abbreviations: ER, estrogen-receptor; aCGH, array comparative genomic hybridization.

FIG. 10 depicts characteristics and treatments of 81 Stage-III series patients, which did not differ from ER-low, HER2-negative patients.

FIG. 11 depicts univariate Cox proportional-hazard regression analysis of the risk of recurrence in the Stage-III patients.

FIG. 12 depicts the association of BRCA1-classification with outcome after HD-chemotherapy and conventional chemotherapy in the stage-III series. Kaplan Meier survival curves according to BRCA1-classification. A) Recurrence Free Survival (RFS) of BRCA1-like patients who had been randomized between HD-chemotherapy or conventional chemotherapy. B) Recurrence Free Survival (RFS) of Sporadic-like patients who had been randomized between HD-chemotherapy or conventional chemotherapy.

FIG. 13 depicts performance of different cut-offs of the BRCA1-probability score using a BAC classifier comprising 427 BAC clones, as disclosed herein, to identify patients with a progression free survival of more than 24 months. A. Positive predictive values and negative predictive values at different cut-offs. B. Receiver operating curve (ROC). Red circle corresponds to cut-off chosen for further analysis.

FIG. 14 depicts Kaplan-Meier curves for progression free survival by BRCA1-like and Sporadic-like classification in the MBC-series. All patients. p-value represents logrank test of equal survival.

FIG. 15 depicts BRCA1 gene expression versus methylation status (p<0.001) in TN tumors.

FIG. 16 depicts BRCA1-like aCGH pattern (p=0.285) in TN tumors.

### DETAILED DESCRIPTION

### Definitions

“Anti-cancer therapy” means any one, or a plurality, of therapies and/or drugs used to treat cancer, or any combinations thereof, including a) homologous recombination deficiency-targeted drugs and/or treatments; and b) drugs or treatments that directly or indirectly cause double strand DNA breaks. This definition includes, without limitation, high dose platinum-based alkylating chemotherapy, platinum compounds, thiotepa, cyclophosphamide, iphosphamide, nitrosureas, nitrogen mustard derivatives, mitomycins, epipodophyllotoxins, camptothecins, anthracyclines, poly(ADP-ribose) polymerase (PARP) inhibitors, ionizing radiation, ABT-888, olaparib (AZT-2281), gemcitabine, CEP-9722, AG014699, AG014699 with Temozolomide, and BSI-201.

“Array” refers to an arrangement, on a substrate surface, of one or a plurality of nucleic acid probes (as defined herein) of predetermined identity. In various embodiments, the sequences of the nucleic acid probes are known. In general, an array comprises a plurality of target elements, each target element comprising one or more nucleic acid probes immobilized on one or more solid surfaces, to which sample nucleic acids can be hybridized. In various embodiments, each individual probe is immobilized to a designated, discrete location (i.e., a defined location or assigned position) on the substrate surface. In various embodiments, each nucleic acid probe is immobilized to a discrete location on an array and each has a sequence that is either specific to, or characteristic of, a particular genomic locus. A nucleic acid probe is specific to, or characteristic of, a genomic locus when it contains a nucleic acid sequence that is unique to that genomic locus. Such a probe preferentially hybridizes to a nucleic acid made from that genomic locus, and not to nucleic acids made from other genomic loci.

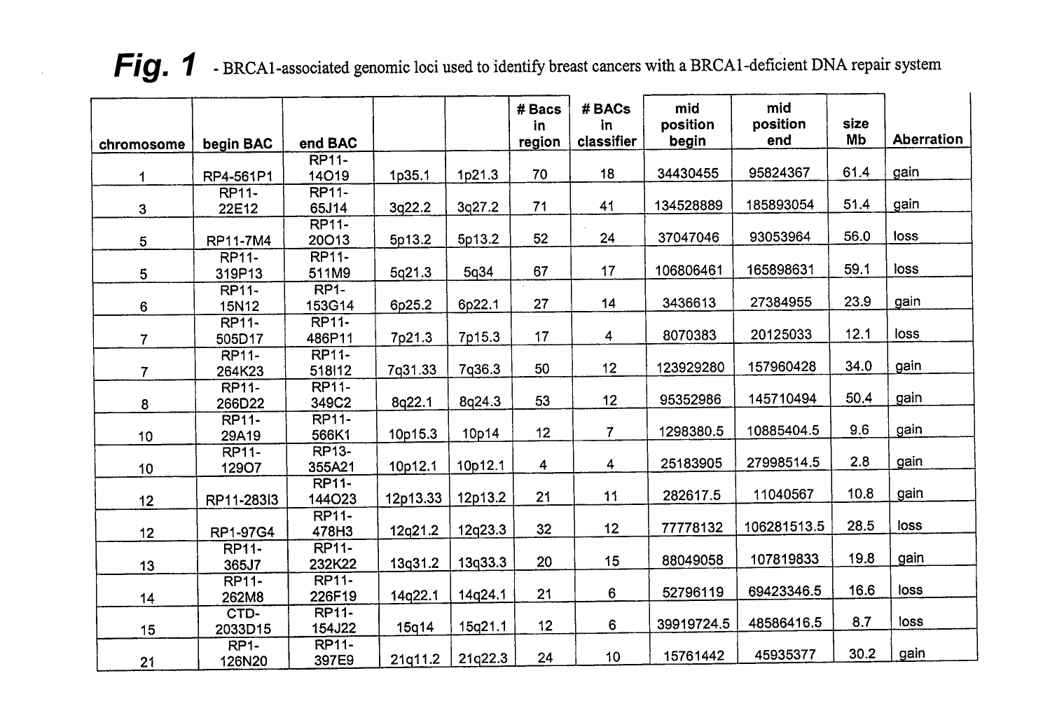

The nucleic acid probes can contain sequence(s) from specific genes or clones. In various embodiments, at least some of the nucleic acid probes contain sequences from any one or more of the specific genomic regions recited in FIG. 1. In various embodiments, at least some of the nucleic acid probes contain sequences of known, reference genes or clones. In various embodiments, the nucleic acid probes in a single array contain both sequences from any one or more of the specific genomic regions recited in FIG. 1 and sequences of known, reference genes or clones.

The probes may be arranged on the substrate in a single density, or in varying densities. The density of each of the probes can be varied to accommodate certain factors such as, for example, the nature of the test sample, the nature of a label used during hybridization, the type of substrate used, and the like. Each probe may comprise a mixture of nucleic acids of varying lengths and, thus, varying sequences. For example, a single probe may contain more than one copy of a cloned nucleic acid, and each copy may be broken into fragments of different lengths. Each length will thus have a different sequence.

The length, sequence and complexity of the nucleic acid probes may be varied. In various embodiments, the length, sequence and complexity are varied to provide optimum hybridization and signal production for a given hybridization procedure, and to provide the required resolution among different genes or genomic locations.

“BRCA1-associated tumor” means a tumor having cells containing a mutation of the BRCA1 locus or a homologous recombination pathway deficiency that directly or indirectly alters BRCA1 activity or function.

“CGH” or “Comparative Genomic Hybridization” refers generally to molecular-cytogenetic techniques for the analysis of copy number changes, gains and/or losses, in the DNA content of a given subject's DNA. CGH can be used to identify chromosomal alterations, such as unbalanced chromosomal changes, in any number of cells including, for example, cancer cells. In various embodiments, CGH is utilized to detect one or more chromosomal amplifications and/or deletions of regions between a test sample and a reference sample.

“Chromosomal locus” refers to a specific, defined portion of a chromosome.

“Genome” refers to all nucleic acid sequences, coding and non-coding, present in each cell type of a subject. The term also includes all naturally occurring or induced variations of these sequences that may be present in a mutant or disease variant of any cell type, including, for example, tumor cells. Genomic DNA and genomic nucleic acids are thus nucleic acids isolated from a nucleus of one or more cells, and include nucleic acids derived from, isolated from, amplified from, or cloned from genomic DNA, as well as synthetic versions of all or any part of a genome.

For example, the human genome consists of approximately 3.0×10⁹ base pairs of DNA organized into 46 distinct chromosomes. The genome of a normal human diploid somatic cell consists of 22 pairs of autosomes (chromosomes 1 to 22) and either chromosomes X and Y (male) or a pair of X chromosomes (female) for a total of 46 chromosomes. A genome of a cancer cell may contain variable numbers of each chromosome in addition to deletions, rearrangements and amplification of any sub-chromosomal region or DNA sequence.

“Genomic locus” refers to a specific, defined portion of a genome.

“HBOC tumors” refers to tumors present in a patient or a group of patients with a high risk for BRCA1-associated breast cancer (patients from Hereditary Breast and Ovarian Cancer families) but who display a negative screen result for BRCA1 and/or BRCA2 mutations. Such patients have a family history that include at least two breast cancer cases and one ovarian cancer case.

“Hybridization” refers to the binding of two single stranded nucleic acids via complementary base pairing. Extensive guides to the hybridization of nucleic acids can be found in: Tijssen, Laboratory Techniques in Biochemistry and Molecular Biology-Hybridization with Nucleic Acid Probes Part I, Ch. 2, “Overview of principles of hybridization and the strategy of nucleic acid probe assays” (1993), Elsevier, N.Y.; and Sambrook et al., Molecular Cloning: A Laboratory Manual (3rd ed.) Vol. 1-3 (2001), Cold Spring Harbor Laboratory, Cold Spring Harbor Press, N.Y. The phrases “hybridizing specifically to”, “specific hybridization”, and “selectively hybridize to”, refer to the preferential binding, duplexing, or hybridizing of a nucleic acid molecule to a particular probe under stringent conditions. The term “stringent conditions” refers to hybridization conditions under which a probe will hybridize preferentially to its target subsequence, and to a lesser extent, or not at all, to other sequences in a mixed population (e.g., a DNA preparation from a tissue biopsy). “Stringent hybridization” and “stringent hybridization wash conditions” are sequence-dependent and are different under different environmental parameters.

Generally, highly stringent hybridization and wash conditions are selected to be about 5° C. lower than the thermal melting point (Tm) for a specific sequence at a defined ionic strength and pH. The Tm is the temperature at which 50% of the target sequence hybridizes to a perfectly matched probe. Very stringent conditions are selected to be equal to the Tm for a particular probe. Often, a high stringency wash is preceded by a low stringency wash to remove background probe signal. An example of stringent hybridization conditions for hybridization of complementary nucleic acids which have more than 100 complementary residues on an array is 42° C. using standard hybridization solutions, with the hybridization being carried out overnight. An example of highly stringent wash conditions is a 0.15 M NaCl wash at 72° C. for 15 minutes. An example of stringent wash conditions is a wash in 0.2× Standard Saline Citrate (SSC) buffer at 65° C. for 15 minutes. An example of a medium stringency wash for a duplex of, for example, more than 100 nucleotides, is 1×SSC at 45° C. for 15 minutes. An example of a low stringency wash for a duplex of, for example, more than 100 nucleotides, is 4× to 6×SSC at 40° C. for 15 minutes.

“Micro-array” refers to an array that is miniaturized so as to require microscopic examination for visual evaluation. In various embodiments, the arrays used in the methods of the present disclosure can be micro-arrays.

“Nucleic acid” refers to a deoxyribonucleotide or ribonucleotide in either single- or double-stranded form and includes all nucleic acids comprising naturally occurring nucleotide bases as well as nucleic acids containing any and/or all analogues of natural nucleotides. This term also includes nucleic acid analogues that are metabolized in a manner similar to naturally occurring nucleotides, but at rates that are improved for the purposes desired. This term also encompasses nucleic-acid-like structures with synthetic backbone analogues including, without limitation, phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs) (see, e.g.: “Oligonucleotides and Analogues, a Practical Approach,” edited by F. Eckstein, IRL Press at Oxford University Press (1991); “Antisense Strategies,” Annals of the New York Academy of Sciences, Volume 600, Eds. Baserga and Denhardt (NYAS 1992); Milligan (1993) J. Med. Chem. 36:1923-1937; and “Antisense Research and Applications” (1993, CRC Press)). PNAs contain non-ionic backbones, such as N-(2-aminoethyl) glycine units. Phosphorothioate linkages are described in: WO 97/03211; WO 96/39154; and Mata (1997) Toxicol. Appl. Pharmacol. 144:189-197. Other synthetic backbones encompassed by this term include methyl-phosphonate linkages or alternating methyl-phosphonate and phosphodiester linkages (Strauss-Soukup (1997) Biochemistry 36: 8692-8698), and benzyl-phosphonate linkages (Samstag (1996) Antisense Nucleic Acid Drug Dev 6: 153-156).

“Probe” or “nucleic acid probe” refer to one or more nucleic acid fragments whose specific hybridization to a sample can be detected. In various embodiments, probes are arranged on a substrate surface in an array. The probe may be unlabelled, or it may contain one or more labels so that its binding to a nucleic acid can be detected. In various embodiments, a probe can be produced from any source of nucleic acids from one or more particular, pre-selected portions of a chromosome including, without limitation, one or more clones, an isolated whole chromosome, an isolated chromosome fragment, or a collection of polymerase chain reaction (PCR) amplification products.

In some embodiments, the probe may be a member of an array of nucleic acids as described in WO 96/17958. Techniques capable of producing high density arrays can also be used for this purpose (see, e.g., Fodor (1991) Science 767-773; Johnston (1998) Curr. Biol. 8: RI 71-RI 74; Schummer (1997) Biotechniques 23: 1087-1092; Kern (1997) Biotechniques 23: 120-124; and U.S. Pat. No. 5,143,854).

The sequence of the probes can be varied. In various embodiments, the probe sequence can be varied to produce probes that are substantially identical to the probes disclosed herein, but that retain the ability to hybridize specifically to the same targets or samples as the probe from which they were derived.

“Reference sample” refers to nucleic acids comprising sequences whose quantity or degree of representation, copy number, and/or sequence identity are known. Such nucleic acids serve as a reference to which one or more test samples are compared.

“Sample” refers to a material, or mixture of materials, containing one or more components of interest. Samples include, but are not limited to, material obtained from an organism and may be directly obtained from a source, such as from a biopsy or from a tumor, or indirectly obtained such as after culturing and/or processing.

“Test sample” refers to nucleic acids comprising sequences whose quantity or degree of representation, copy number, and/or sequence identity are unknown. In various embodiments, the present disclosure is directed to the detection of the quantity or degree of representation, copy number, and/or sequence identity of one or more test samples.

Reference is now made in detail to certain embodiments of arrays and methods. The disclosed embodiments are not intended to be limiting of the claims. To the contrary, the claims are intended to cover all alternatives, modifications, and equivalents.

### Arrays, Micro-Arrays and Probes

In various aspects, the present disclosure relates to the determination of copy number changes in the DNA content of a given test sample, as compared to one or more reference samples. In some embodiments, the copy number changes comprise gains or increases in the DNA content of a test sample. In some embodiments, the copy number changes comprise losses or decreases in the DNA content of a test sample. In some embodiments, the copy number changes comprise both gains or increases and losses or decreases in the DNA content of a test sample.

Copy number changes can be determined by hybridizations that are performed on a solid support. For example, probes that selectively hybridize to specific chromosomal regions can be spotted onto a surface. In various aspects, the spots of probes are placed in an ordered pattern, or array, and the pattern is recorded to facilitate correlation of results. Once an array is generated, one or more test samples can be hybridized to the array. In various aspects, arrays comprise a plurality of nucleic acid probes immobilized to discrete spots (i.e., defined locations or assigned positions) on a substrate surface.

Thus, in several aspects, copy number changes of genomic loci are analyzed in an array-based approach. In some embodiments, copy number changes of genomic loci are analyzed using comparative genomic hybridization. In some embodiments, copy number changes of genomic loci are analyzed using array-based comparative genomic hybridization.

Any of a variety of arrays may be used. A number of arrays are commercially available for use from Vysis Corporation (Downers Grove, Ill.), Spectral Genomics Inc. (Houston, Tex.), and Affymetrix Inc. (Santa Clara, Calif.). Arrays can also be custom made for one or more hybridizations.

Methods of making and using arrays are well known in the art (see, e.g., Kern et al., Biotechniques (1997), 23:120-124; Schummer et al., Biotechniques (1997), 23:1087-1092; Solinas-Toldo et al., Genes, Chromosomes & Cancer (1997), 20: 399-407; Johnston, Curr. Biol. (1998), 8: RI 71-RI 74; Bowtell, Nature Gen. (1999), Supp. 21:25-32; Watson et al., Biol. Psychiatry (1999), 45: 533-543; Freeman et al., Biotechniques (2000), 29: 1042-1046 and 1048-1055; Lockhart et al., Nature (2000), 405: 827-836; Cuzin, Transfus. Clin. Biol. (2001), 8:291-296; Zarrinkar et al., Genome Res. (2001), 11: 1256-1261; Gabig et al., Acta Biochim. Pol. (2001), 48: 615-622; and Cheung et al., Nature (2001), 40: 953-958; see also, e.g., U.S. Pat. Nos. 5,143,854; 5,434,049; 5,556,752; 5,632,957; 5,700,637; 5,744,305; 5,770,456; 5,800,992; 5,807,522; 5,830,645; 5,856,174; 5,959,098; 5,965,452; 6,013,440; 6,022,963; 6,045,996; 6,048,695; 6,054,270; 6,258,606; 6,261,776; 6,277,489; 6,277,628; 6,365,349; 6,387,626; 6,458,584; 6,503,711; 6,516,276; 6,521,465; 6,558,907; 6,562,565; 6,576,424; 6,587,579; 6,589,726; 6,594,432; 6,599,693; 6,600,031; and 6,613,893).

Substrate surfaces suitable for use in the generation of an array can be made of any rigid, semi-rigid or flexible material that allows for direct or indirect attachment (i.e., immobilization) of nucleic acid probes to the substrate surface. Suitable materials include, without limitation, cellulose (see, e.g., U.S. Pat. No. 5,068,269), cellulose acetate (see, e.g., U.S. Pat. No. 6,048,457), nitrocellulose, glass (see, e.g., U.S. Pat. No. 5,843,767), quartz and/or other crystalline substrates such as gallium arsenide, silicones (see, e.g., U.S. Pat. No. 6,096,817), plastics and plastic copolymers (see, e.g., U.S. Pat. Nos. 4,355,153; 4,652,613; and 6,024,872), membranes and gels (see, e.g., U.S. Pat. No. 5,795,557), and paramagnetic or supramagnetic microparticles (see, e.g., U.S. Pat. No. 5,939,261). When fluorescence is to be detected, arrays comprising cyclo-olefin polymers may be used (see, e.g., U.S. Pat. No. 6,063,338). The presence of reactive functional chemical groups (such as, for example, hydroxyl, carboxyl, and amino groups) present on the surface of the substrate material can be used to directly or indirectly attach nucleic acid probes to the substrate surface.

More than one copy of each nucleic acid probe may be spotted onto an array. For example, each nucleic acid probe may be spotted onto an array once, in duplicate, in triplicate, or more, depending on the desired application. Multiple spots of the same probe allows for assessment of the reproducibility of the results obtained.

Related nucleic acid probes may also be grouped together, in probe elements, on an array. For example, a single probe element may include a plurality of spots of related nucleic acid probes, which are of different lengths but that comprise substantially the same sequence or that are derived from the sequence of a specific genomic locus. Alternatively, a single probe element may include a plurality of spots of related nucleic acid probes that are fragments of different lengths resulting from digestion of more than one copy of a cloned nucleic acid. An array may contain a plurality of probe elements and probe elements may be arranged on an array at different densities.

Array-immobilized nucleic acid probes may be nucleic acids that contain sequences from genes (e.g., from a genomic library) including, for example, sequences that collectively cover a substantially complete genome, or any one or more subsets of a genome. In various embodiments, the sequences of the nucleic acid probes on an array comprise those for which comparative copy number information is desired. In some embodiments, to obtain DNA sequence copy number information across an entire genome, an array comprising nucleic acid probes covering a whole genome or a substantially complete genome is used. In some embodiments, at least one relevant genomic locus has been determined and is used in an array, such that there is no need for genome-wide hybridization. In some embodiments, a plurality of relevant genomic loci have been determined and are used in an array, such that there is no need for genome-wide hybridization. In some embodiments, the array comprises a plurality of specific nucleic acid probes that originate from a discrete set of genes or genomic loci and whose copy number, in association with the type of condition or tumor is to be tested, is known. Additionally, the array may comprise nucleic acid probes that will serve as positive or negative controls. In some embodiments, the array comprises a plurality of nucleic acid sequences derived from karyotypically normal genomes.

The probes may be generated by any number of known techniques (see, e.g., Tijssen, Laboratory Techniques in Biochemistry and Molecular Biology-Hybridization with Nucleic Acid Probes Part I, Ch. 2, “Overview of principles of hybridization and the strategy of nucleic acid probe assays” (1993), Elsevier, N.Y.; Sambrook et al., Molecular Cloning: A Laboratory Manual (3rd ed.) Vol. 1-3 (2001), Cold Spring Harbor Laboratory, Cold Spring Harbor Press, N.Y.; Innis (Ed.) “PCR Strategies” (1995), Academic Press: New York, N.Y.; and Ausubel (Ed.), “Short Protocols in Molecular Biology” 5th Ed. (2002), John Wiley & Sons). Nucleic acid probes may be obtained and manipulated by cloning into various vehicles. They may be screened and re-cloned or amplified from any source of genomic DNA.

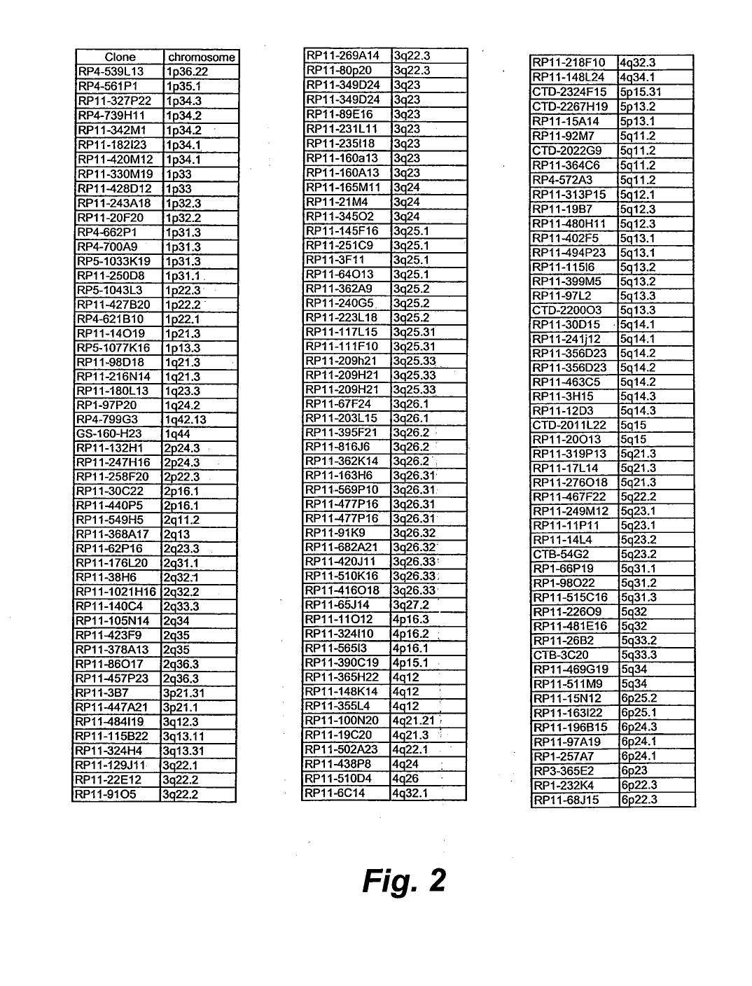

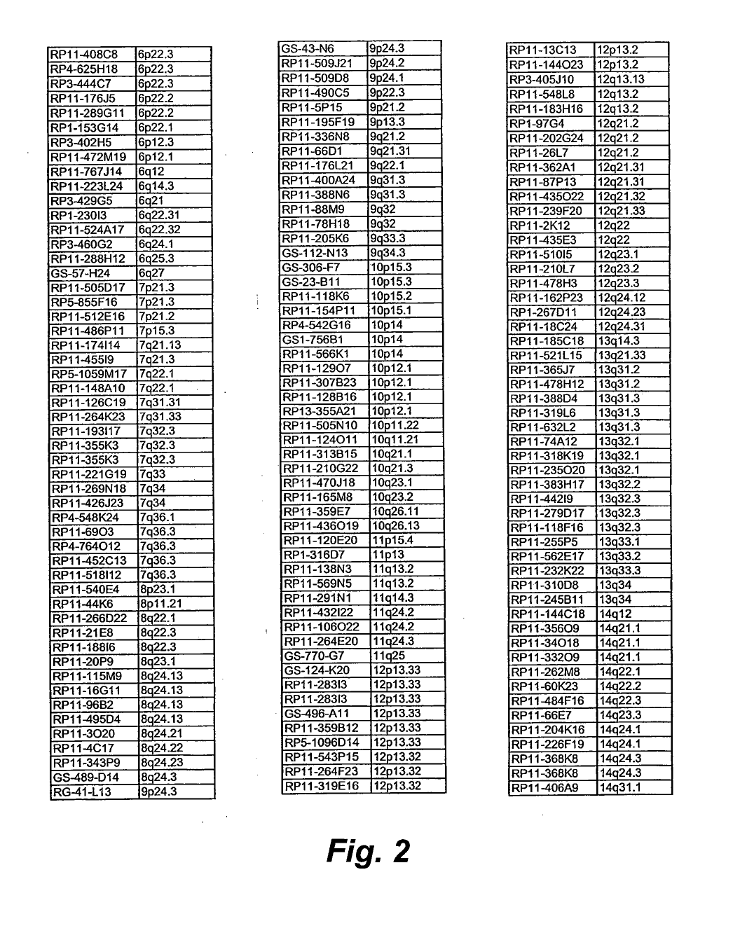

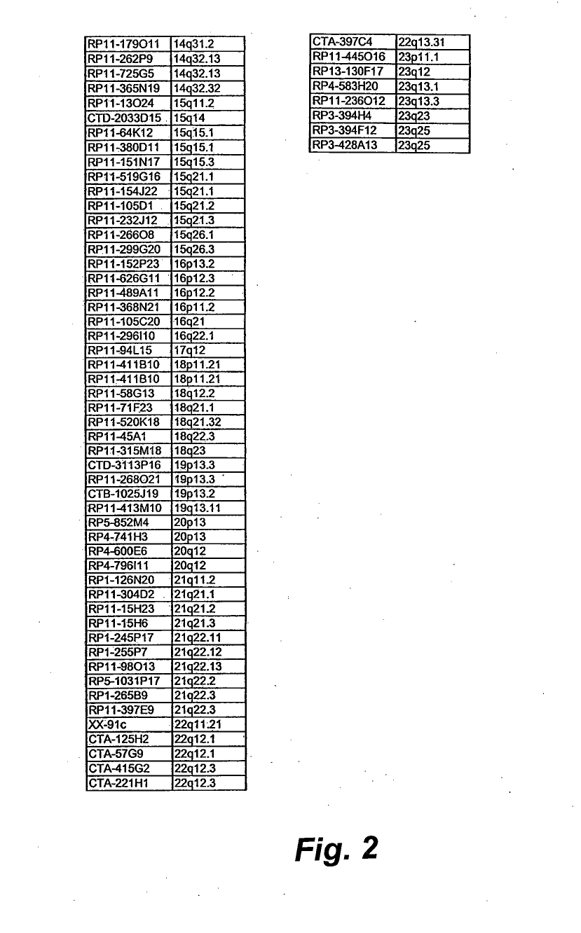

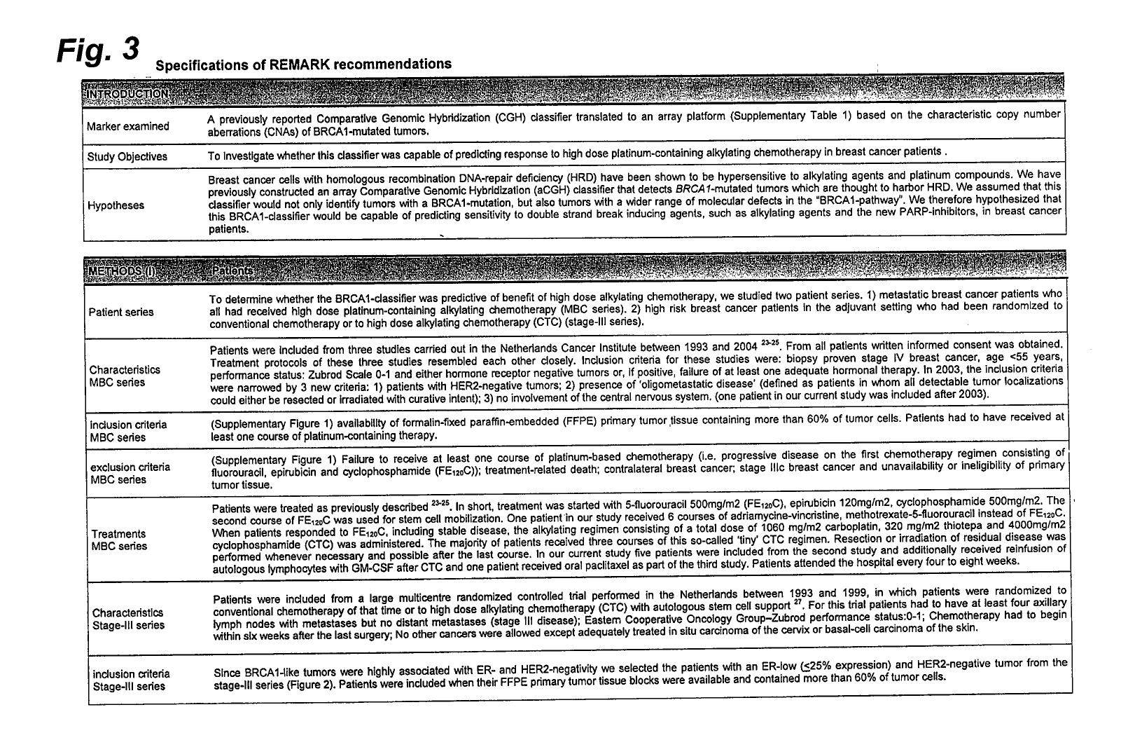

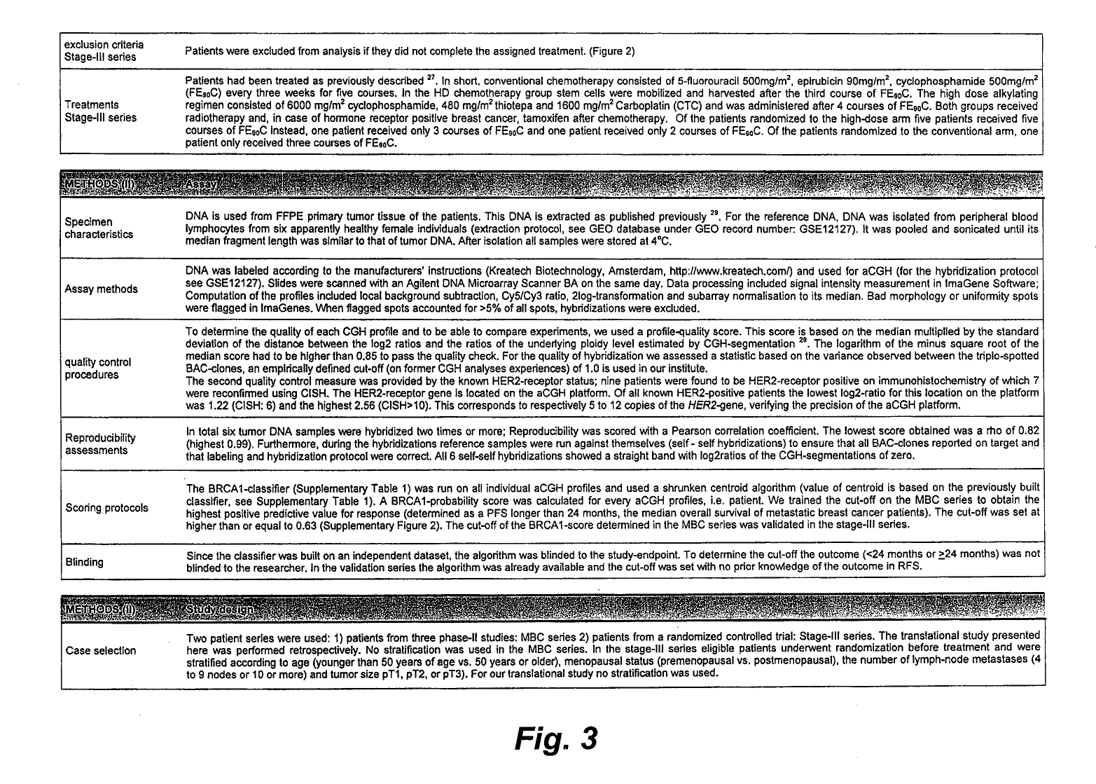

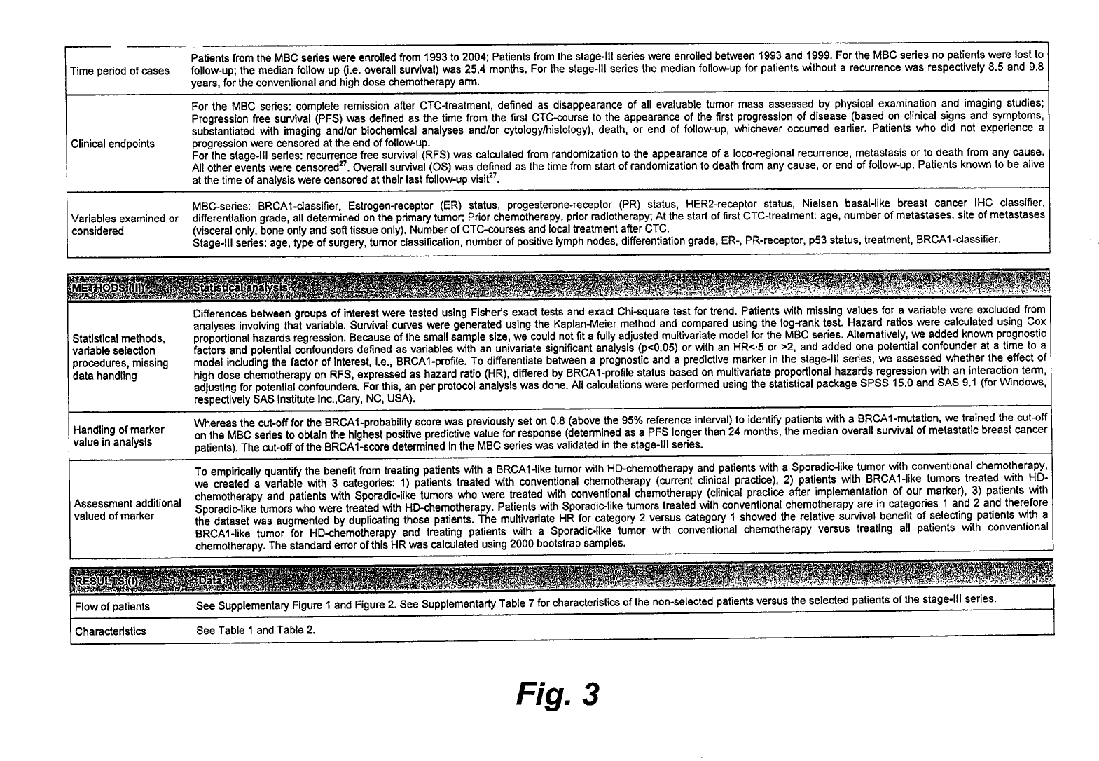

Nucleic acid probes may also be obtained and manipulated by cloning into vehicles including, for example, recombinant viruses, cosmids, or plasmids. Nucleic acid probes may also be synthesized in vitro by chemical techniques (see, e.g., Nucleic Acids Res. (1997), 25: 3440-3444; Blommers et al., Biochemistry (1994), 33: 7886-7896; and Frenkel et al., Free Radic. Biol. Med. (1995), 19: 373-380). Probes may vary in size from synthetic oligonucleotide probes and/or PCR-type amplification primers of a few base pairs in length to artificial chromosomes of more than 1 megabases in length. In various embodiments, probes comprise at least 10, at least 12, at least 15, at least 18, at least 20, at least 22, at least 30, at least 50 or at least 100 contiguous nucleotides of a sequence present in a BAC clone set forth in FIG. 2. In some embodiments, probes comprise a sequence that is unique in a genome. In some embodiments, probes comprise a sequence that is unique in the human genome.

Probes may be obtained from any number of commercial sources. For instance, several P1 clones are available from the DuPont P1 library (see, e.g., Shepard et al., Proc. Natl. Acad. Sci. USA (1994), 92: 2629), and available commercially from Incyte Corporation (Wilmington, Del.). Various libraries spanning entire chromosomes are available commercially from Clontech Laboratories, Inc. (Mountain View, Calif.), or from the Los Alamos National Laboratory (Los Alamos, Calif.). In various aspects, the present disclosure relates to the use of the human 3600 BAC/PAC genomic clone set, covering the full human genome at 1 Mb spacing, obtained from the Wellcome Trust Sanger Institute (Hinxton, Cambridge, UK).

In some embodiments, the nucleic acid probes are derived from mammalian artificial chromosomes (MACs) and/or human artificial chromosomes (HACs), which can contain inserts from about 5 to 400 kilobases (kb) (see, e.g., Roush, Science (1997), 276: 38-39; Rosenfeld, Nat. Genet. (1997), 15: 333-335; Ascenzioni et al., Cancer Lett. (1997), 118: 135-142; Kuroiwa et al., Nat. Biotechnol. (2000), 18: 1086-1090; Meija et al., Am. J. Hum. Genet. (2001), 69: 315-326; and Auriche et al., EMBO Rep. (2001), 2: 102-107).

In some embodiments, the nucleic acid probes are derived from satellite artificial chromosomes or satellite DNA-based artificial chromosomes (SATACs). SATACs can be produced by inducing de novo chromosome formation in cells of varying mammalian species (see, e.g., Warburton et al., Nature (1997), 386: 553-555; Csonka et al., J. Cell. Sci. (2000), 113: 3207-3216; and Hadlaczky, Curr. Opin. Mol. Ther. (2001), 3: 125-132).

In some embodiments, the nucleic acid probes are derived from yeast artificial chromosomes (YACs), 0.2-1 megabses in size. YACs have been used for many years for the stable propagation of genomic fragments of up to one million base pairs in size (see, e.g., Feingold et al., Proc. Natl. Acad. Sci. USA (1990), 87:8637-8641; Adam et al., Plant J. (1997), 11: 1349-1358; Tucker et al., Gene (1997), 199: 25-30; and Zeschnigk et al., Nucleic Acids Res. (1999), 27: E30).

In some embodiments, the nucleic acid probes are derived from bacterial artificial chromosomes (BACs) up to 300 kb in size. BACs are based on the E. coli F factor plasmid system and are typically easy to manipulate and purify in microgram quantities (see, e.g., Asakawa et al., Gene (1997), 191: 69-79; and Cao et al., Genome Res. (1999), 9: 763-774).

In some embodiments, the nucleic acid probes are derived from P1 artificial chromosomes (PACs), about 70-100 kb in size. PACs are bacteriophage P1-derived vectors (see, e.g., Ioannou et al., Nature Genet. (1994), 6: 84-89; Boren et al., Genome Res. (1996), 6: 1123-1130; Nothwang et al., Genomics (1997), 41: 370-378; Reid et al., Genomics (1997), 43: 366-375; and Woon et al., Genomics (1998), 50: 306-316).

In some embodiments, the array comprises a series of separate wells or chambers on the substrate surface, into which probes may be immobilized as described herein. The probes can be immobilized in the separate wells or chambers and hybridization can take place within the wells or chambers. In various embodiments, the arrays can be selected from chips, microfluidic chips, microtiter plates, Petri dishes, and centrifuge tubes. Robotic equipment has been developed for these types of arrays that permit automated delivery of reagents into the separate wells or chambers which allow the amount of the reagents used per hybridization to be sharply reduced. Examples of chip and microfluidic chip techniques can be found, for example, in U.S. Pat. No. 5,800,690; Orchid, “Running on Parallel Lines” New Scientist (1997); McCormick et al., Anal. Chem. (1997), 69:2626-30; and Turgeon, “The Lab of the Future on CD-ROM?” Medical Laboratory Management Report. December 1997, p. 1.

### BRCA1 Arrays

An array comparative genomic hybridization (aCGH) profile that distinguishes BRCA1-mutated breast cancers from sporadic breast cancers has been identified and is disclosed herein. In various aspects, the present disclosure relates to the use of a BRCA1 array comprising this unique BRCA1 aCGH profile to identify breast cancers with a homologous recombination deficiency due to a defect in BRCA1 or in the homologous recombination pathway which results in a BRCA1-like phenotype, and to thus identify patients, from whom the cancers have been excised, who will be highly sensitive to certain anti-cancer therapy. Therefore, in various aspects, the present disclosure relates to the use of a BRCA1 array comprising this BRCA1 aCGH profile to prospectively optimize the therapeutic efficacy of anti-cancer therapy in an individual subject by detecting phenotypic genetic traits associated with deficiencies in the BRCA1 gene or in the homologous recombination pathway which results in a BRCA1-like phenotype.

In various embodiments, a BRCA1 array comprising a BRCA1 aCGH profile for identifying individual subjects who will experience a therapeutic benefit from anti-cancer therapy is provided. In some embodiments, a BRCA1 array is used to detect BRCA1-associated genomic copy number variations in a test sample, as compared to a reference sample, at one, or a plurality, of the genomic loci selected from 1p35-21, 3q22-27, 5p13, 5q21-34, 6p25-22, 7p21-15, 7q31-36, 8q22-24, 10p15-14, 10p12, 12p13, 12q21-23, 13q31-33, 14q22-24, 15q14-21 and 21q11-22. In some embodiments, a BRCA1 array is used to detect an increase in genomic copy numbers in a test sample, as compared to a reference sample, in any one, or a plurality, of the genomic loci selected from 1p35-21, 3q22-27, 6p25-22, 7q31-36, 8q22-24, 10p15-14, 10p12, 12p13, 13q31-33, and 21q11-22. In some embodiments, a BRCA1 array is used to detect a decrease in genomic copy numbers in a test sample, as compared to a reference sample, in any one, or a plurality, of the genomic loci selected from 5p13, 5q21-34, 7p21-15, 12q21-23, 14q22-24 and 15q14-21. In each of the aforementioned embodiments, detection of BRCA1-associated genomic copy number variations classifies the test sample as from a BRCA1-associated tumor and classifies the subject from whom the test sample was excised as an individual who will experience a therapeutic benefit from anti-cancer therapy.

The genomic loci may be detected individually, or in any combination of two or more loci. In some embodiments, a BRCA1 array is used that is capable of detecting BRCA1-associated genomic copy number variations in all 16 of the above-listed chromosomal loci. In some embodiments, a BRCA1 array is used that is capable of detecting BRCA1-associated genomic copy number variations in a number of genomic loci selected from greater than 1, greater than 2, greater than 3, greater than 4, greater than 5, greater than 6, greater than 7, greater than 8, greater than 9, greater than 10, greater than 11, greater than 12, greater than 13, greater than 14 and greater than 15. In some embodiments, a BRCA1 array is used that is capable of detecting BRCA1-associated genomic copy number variations in a number of genomic loci selected from less than 16, less than 15, less than 14, less than 13, less than 12, less than 11, less than 10, less than 9, less than 8, less than 7, less than 6, less than 5, less than 4, less than 3, and less than 2. In some embodiments, a BRCA1 array is used that is capable of detecting BRCA1-associated genomic copy number variations in all 16 of the BRCA1-associated genomic loci set forth in FIG. 1. In each of the aforementioned embodiments, detection of BRCA1-associated genomic copy number variations classifies the test sample as from a BRCA1-associated tumor and classifies the subject from whom the test sample was excised as an individual who will experience a therapeutic benefit from anti-cancer therapy.

The BRCA1 arrays comprise at least one probe. In various embodiments, the BRCA1 arrays comprise a plurality of probes. In some embodiments, the BRCA1 arrays comprise a plurality of probes, wherein the probes comprise nucleic acid sequences derived from BAC clones. The BRCA1-associated genomic loci set forth in FIG. 1 are bounded by the BAC probes set forth in FIG. 2. In some embodiments, arrays capable of detecting BRCA1-associated genomic copy number variations comprise at least one, or a plurality, of probes derived from the BAC clones of FIG. 2. The BAC clones set forth in FIG. 2 are not intended to be limiting in any way, and other probes within the BRCA1-associated genomic loci of FIG. 1 can also be used in the BRCA1 arrays. In some embodiments, arrays capable of detecting BRCA1-associated genomic copy number variations comprise all 371 of the BAC clones of FIG. 2. In some embodiments, arrays capable of detecting BRCA1-associated genomic copy number variations comprise a number of BAC clones of FIG. 2 selected from greater than 1, greater than 10, greater than 20, greater than 25, greater than 50, greater than 75, greater than 100, greater than 125, greater than 150, greater than 175, greater than 200, greater than 225, greater than 250, greater than 275, greater than 300, greater than 325 and greater than 350. In some embodiments, arrays capable of detecting BRCA1-associated genomic copy number variations comprise a number of BAC clones of FIG. 2 selected from less than 371, less than 350, less than 325, less than 300, less than 275, less than 250, less than 225, less than 200, less than 175, less than 150, less than 125, less than 100, less than 75, less than 50, less than 25, less than 20, and less than 10.

In some embodiments, a BRCA1 array capable of detecting BRCA1-associated genomic copy number variations comprises at least one, or a plurality, of probes that independently hybridize to at least one, or a plurality, of the genomic loci selected from 1p35-21, 3q22-27, 5p13, 5q21-34, 6p25-22, 7p21-15, 7q31-36, 8q22-24, 10p15-14, 10p12, 12p13, 12q21-23, 13q31-33, 14q22-24, 15q14-21 and 21q11-22. In these embodiments, the probes are as defined above and/or may be obtained in methods as described above.

In some embodiments, BRCA1 arrays capable of detecting BRCA1-associated genomic copy number variations comprise at least one, or a plurality, of probes, wherein the probes comprise at least one, or a plurality, of the distinct BAC clones of FIG. 2. In some embodiments, BRCA1 arrays capable of detecting BRCA1-associated genomic copy number variations comprise at least one, or a plurality, of probes, wherein the probes comprise at least one, or a plurality, of the BAC clones of FIG. 2, and wherein the probes specifically hybridize to at least 1, at least 2, at least 3, at least 4, at least 5, at least 6, at least 7, at least 8, at least 9, at least 10, at least 11, at least 12, at least 13, at least 14 or at least 15 of the genomic loci set forth in FIG. 1. In some embodiments, BRCA1 arrays capable of detecting BRCA1-associated genomic copy number variations comprise a plurality of probes, wherein the nucleic acid sequences of the probes are unique to the genomic loci set forth in FIG. 1. In some embodiments, BRCA1 arrays capable of detecting BRCA1-associated genomic copy number variations comprise a plurality of probes, wherein the probes comprise a plurality of BAC clones specific to all of the genomic loci set forth in FIG. 1. In some embodiments, BRCA1 arrays capable of detecting BRCA1-associated genomic copy number variations comprise at least one, or a plurality, of probes, wherein the probes comprise at least 1, at least 2, at least 3, at least 4, at least 5, at least 6, at least 7, at least 8, at least 9, at least 10, at least 15, at least 20, at least 25, at least 50, at least 75, at least 100, at least 125, at least 150, at least 175, at least 200, at least 225, at least 250, at least 275, at least 300, at least 325 or at least 350 of the distinct BAC clones of FIG. 2.

In various embodiments, BRCA1 arrays capable of detecting BRCA1-associated genomic copy number variations that comprise at least one, or a plurality, of probes, and/or that comprise at least one, or a plurality, of distinct BAC clones, allow for the individual analysis of at least one, or a plurality, of distinct genomic loci. Therefore, in some embodiments, the probes, and/or the distinct BAC clones, capable of detecting BRCA1-associated genomic copy number variations are arranged on the BRCA1 arrays in a positionally-addressable manner.

In various embodiments, BRCA1 arrays capable of detecting BRCA1-associated genomic copy number variations comprise at least one, or a plurality, of distinct BAC clones, wherein the distinct BAC clones represent at least 1, at least 2, at least 3, at least 4, at least 5, at least 6, at least 7, at least 8, at least 9, at least 10, at least 11, at least 12, at least 13, at least 14 or at least 15 of the genomic loci set forth in FIG. 1. In various embodiments, BRCA1 arrays capable of detecting BRCA1-associated genomic copy number variations comprise at least one, or a plurality, of distinct BAC clones, wherein the distinct BAC clones represent all 16 of the genomic loci set forth in FIG. 1.

### Array Comparative Genomic Hybridization

In various aspects, the present disclosure relates to the analysis of tumor cell samples by array-based comparative genomic hybridization. Array comparative genomic hybridization (aCGH) is a technique that is used to detect genomic copy number variations at a higher level of resolution than chromosome-based comparative genomic hybridization. In aCGH, nucleic acids from a test sample and nucleic acids from a reference sample are labelled differentially. The test sample and the reference sample are then and hybridized to an array comprising a plurality of probes. The ratio of the signal intensity of the test sample to that of the reference sample is then calculated, to measure the copy number changes for a particular location in the genome. The difference in the signal ratio determines whether the total copy numbers of the nucleic acids in the test sample are increased or decreased as compared to the reference sample. The test sample and the reference sample may be hybridized to the array separately or they may be mixed together and hybridized simultaneously. Exemplary methods of performing aCGH can be found, for example, in U.S. Pat. Nos. 5,635,351; 5,665,549; 5,721,098; 5,830,645; 5,856,097; 5,965,362; 5,976,790; 6,159,685; 6,197,501; and 6,335,167; European Patent Nos. EP 1 134 293 and EP 1 026 260; van Beers et al., Brit. J. Cancer (2006), 20; Joosse et al., BMC Cancer (2007), 7:43; Pinkel et al., Nat. Genet. (1998), 20: 207-211; Pollack et al., Nat. Genet. (1999), 23: 41-46; and Cooper, Breast Cancer Res. (2001), 3: 158-175.

Samples that are labelled differentially are labelled such that one of the two samples is labelled with a first detectable agent and the other of the two samples is labelled with a second detectable agent, wherein the first detectable agent and the second detectable agent produce distinguishable signals. Detectable agents that produce distinguishable signals can include, for example, matched pairs of fluorescent dyes.

In some embodiments, the methods of the present disclosure comprise analyzing at least one test sample of tumor DNA from a subject by array-based comparative genomic hybridization to obtain information relating to the copy number aberrations present in the sample(s), if any; based on the information obtained, classifying the tumor as a BRCA1-associated tumor or a sporadic tumor; and, based on the classification, optimizing the therapeutic efficacy of anti-cancer therapy for the subject by predicting the subject's prospective response to anti-cancer therapy.

Information relating to the copy number aberrations present in a sample can include, for example, a gain of genetic material at one or more genomic loci, a loss of genetic material at one or more genomic loci, chromosomal abnormalities at one or more genomic loci, and genome copy number changes at one or more genomic loci. This information is obtained by analyzing the difference in signal intensity between the test sample and a reference sample at one or more genomic loci. The analysis can be performed using any of a variety of methods, means and variations thereof for carrying out array-based comparative genomic hybridization.

In various embodiments, the reference sample is a nucleic acid sample that is representative of a normal, non-diseased state, for example a non-tumor/non-cancer cell, and contains a normal amount of copy numbers of the complement of the genomic loci being tested. The reference sample may be derived from a genomic nucleic acid sample from a normal and/or healthy individual or from a pool of such individuals. In various embodiments, the reference sample does not comprise any tumor or cancerous nucleic acids. In some embodiments, the reference sample is derived from a pool of female subjects. In some embodiments, the reference sample comprises pooled genomic DNA isolated from tissue samples (e.g. lymphocytes) from a plurality (e.g. at least 4-10) of healthy female subjects. In some embodiments, the reference sample comprises an artificially-generated population of nucleic acids designed to approximate the copy number level from each tested genomic region, or fragments of each tested genomic region. In some embodiments, the reference sample is derived from normal, non-cancerous cell lines or from cell line samples.

Test samples may be obtained from a biological source comprising tumor cells, and reference samples may be obtained from a biological source comprising normal reference cells, by any suitable method of nucleic acid isolation and/or extraction. In various aspects, the test sample and the reference sample are DNA. Methods of DNA extraction are well known in the art. A classical DNA isolation protocol is based on extraction using organic solvents, such as a mixture of phenol and chloroform, followed by precipitation with ethanol (see, e.g., Sambrook et al., supra). Other methods include salting out DNA extraction, trimethylammonium bromide salt extraction, and guanidinium thiocyanate extraction. Additionally, there are numerous DNA extraction kits that are commercially available from, for example, BD Biosciences Clontech (Palo Alto, Calif.), Epicentre Technologies (Madison, Wis.), Gentra Systems, Inc. (Minneapolis, Minn.), MicroProbe Corp. (Bothell, Wash.), Organon Teknika (Durham, N.C.), and Qiagen Inc. (Valencia, Calif.).

The test samples and the reference samples may be differentially labelled with any detectable agents or moieties. In various embodiments, the detectable agents or moieties are selected such that they generate signals that can be readily measured and such that the intensity of the signals is proportional to the amount of labelled nucleic acids present in the sample. In various embodiments, the detectable agents or moieties are selected such that they generate localized signals, thereby allowing resolution of the signals from each spot on an array.

Methods for labeling nucleic acids are well-known in the art. For exemplary reviews of labeling protocols, label detection techniques and recent developments in the field, see: Kricka, Ann. Clin. Biochem. (2002), 39: 114-129; van Gijlswijk et al., Expert Rev. Mol. Diagn. (2001), 1:81-91; and Joos et al., J. Biotechnol. (1994), 35: 135-153. Standard nucleic acid labeling methods include: incorporation of radioactive agents, direct attachment of fluorescent dyes or of enzymes, chemical modification of nucleic acids to make them detectable immunochemically or by other affinity reactions, and enzyme-mediated labeling methods including, without limitation, random priming, nick translation, PCR and tailing with terminal transferase. Other suitable labeling methods include psoralen-biotin, photoreactive azido derivatives, and DNA alkylating agents. In various embodiments, test sample and reference sample nucleic acids are labelled by Universal Linkage System, which is based on the reaction of monoreactive cisplatin derivatives with the N7 position of guanine moieties in DNA (see, e.g., Heetebrij et al., Cytogenet. Cell. Genet. (1999), 87: 47-52).

Any of a wide variety of detectable agents or moieties can be used to label test and/or reference samples. Suitable detectable agents or moieties include, but are not limited to: various ligands; radionuclides such as, for example, ³²P, ³⁵S, ³H, ¹⁴C, ¹²⁵I, ¹³¹I, and others; fluorescent dyes; chemiluminescent agents such as, for example, acridinium esters, stabilized dioxetanes, and others; microparticles such as, for example, quantum dots, nanocrystals, phosphors and others; enzymes such as, for example, those used in an ELISA, horseradish peroxidase, beta-galactosidase, luciferase, alkaline phosphatase and others; colorimetric labels such as, for example, dyes, colloidal gold and others; magnetic labels such as, for example, Dynabeads™; and biotin, dioxigenin or other haptens and proteins for which antisera or monoclonal antibodies are available.

In some embodiments, the test samples and the reference samples are labelled with fluorescent dyes. Suitable fluorescent dyes include, without limitation, Cy-3, Cy-5, Texas red, FITC, Spectrum Red, Spectrum Green, phycoerythrin, rhodamine, and fluorescein, as well as equivalents, analogues and/or derivatives thereof. In some embodiments, the fluorescent dyes selected display a high molar absorption coefficient, high fluorescence quantum yield, and photostability. In some embodiments, the fluorescent dyes exhibit absorption and emission wavelengths in the visible spectrum (i.e., between 400 nm and 750 nm) rather than in the ultraviolet range of the spectrum (i.e., lower than 400 nm). In some embodiments, the fluorescent dyes are Cy-3 (3-N,N′-diethyltetramethylindo-dicarbocyanine) and Cy-5 (5-N,N′-diethyltetramethylindo-dicarbocyanine). Cy-3 and Cy-5 form a matched pair of fluorescent labels that are compatible with most fluorescence detection systems for array-based instruments. In some embodiments, the fluorescent dyes are Spectrum Red and Spectrum Green.

A key component of aCGH is the hybridization of a test sample and a reference sample to an array. Exemplary hybridization and wash protocols are described, for example, in Sambrook et al. (2001), supra; Tijssen (1993), supra; and Anderson (Ed.), “Nucleic Acid Hybridization” (1999), Springer Verlag: New York, N.Y. In some embodiments, the hybridization protocols used for aCGH are those of Pinkel et al., Nature Genetics (1998), 20:207-211. In some embodiments, the hybridization protocols used for aCGH are those of Kallioniemi, Proc. Natl. Acad. Sci. USA (1992), 89:5321-5325.

Methods of optimizing hybridization conditions are well known in the art (see, e.g., Tijssen, (1993), supra). To create competitive hybridization conditions, the array may be contacted simultaneously with differentially labelled nucleic acid fragments of the test sample and the reference sample. This may be done by, for example, mixing the labelled test sample and the labelled reference sample together to form a hybridization mixture, and contacting the array with the mixture.

The specificity of hybridization may be enhanced by inhibiting repetitive sequences. In some embodiments, repetitive sequences (e.g., Alu sequences, L1 sequences, satellite sequences, MRE sequences, simple homo-nucleotide tracts, and/or simple oligonucleotide tracts) present in the nucleic acids of the test sample, reference sample and/or probes are either removed, or their hybridization capacity is disabled. Removing repetitive sequences or disabling their hybridization capacity can be accomplished using any of a variety of well-known methods. These methods include, but are not limited to, removing repetitive sequences by hybridization to specific nucleic acid sequences immobilized to a solid support (see, e.g., Brison et al., Mol. Cell. Biol. (1982), 2: 578-587); suppressing the production of repetitive sequences by PCR amplification using adequately designed PCR primers; inhibiting the hybridization capacity of highly repeated sequences by self-reassociation (see, e.g., Britten et al., Methods of Enzymology (1974), 29: 363-418); or removing repetitive sequences using hydroxyapatite which is commercially available from a number of sources including, for example, Bio-Rad Laboratories, Richmond, Va. In some embodiments, the hybridization capacity of highly repeated sequences in a test sample and/or in a reference sample is competitively inhibited by including, in the hybridization mixture, unlabelled blocking nucleic acids. The unlabelled blocking nucleic acids are therefore mixed with the hybridization mixture, and thus with a test sample and a reference sample, before the mixture is contacted with an array. The unlabelled blocking nucleic acids act as a competitor for the highly repeated sequences and bind to them before the hybridization mixture is contacted with an array. Therefore, the unlabelled blocking nucleic acids prevent labelled repetitive sequences from binding to any highly repetitive sequences of the nucleic acid probes, thus decreasing the amount of background signal present in a given hybridization. In some embodiments, the unlabelled blocking nucleic acids are Human Cot-1 DNA. Human Cot-1 DNA is commercially available from a number of sources including, for example, Gibco/BRL Life Technologies (Gaithersburg, Md.).

Once hybridization is complete, the ratio of the signal intensity of the test sample as compared to the signal intensity of the reference sample is calculated. This calculation quantifies the amount of copy number aberrations present in the genomic DNA of the test sample, if any. In some embodiments, this calculation is carried out quantitatively or semi-quantitatively. In several aspects, it is not necessary to determine the exact copy number aberrations present in the genomic loci tested, as detection of an aberration, i.e. a gain or loss of genetic material, from the copy number in normal, non-cancerous genomic DNA is indicative of the presence of a disease state and is thus sufficient. Therefore, in several embodiments the quantification of the amount of copy number aberrations present in the genomic DNA of a test sample comprises an estimation of the copy number aberrations, as a semi-quantitative or relative measure usually suffices to predict the presence of a disease state and thus prospectively direct the determination of therapy for a subject.

Quantitative techniques may be used to determine the copy number aberrations per cell present in a test sample. Several quantitative and semi-quantitative techniques to determine copy number aberrations exist including, for example, semi-quantitative PCR analysis or quantitative real-time PCR. The Polymerase Chain Reaction (PCR) per se is not a quantitative technique, however PCR-based methods have been developed that are quantitative or semi-quantitative in that they give a reasonable estimate of original copy numbers, within certain limits. Examples of such PCR techniques include, for example, quantitative PCR and quantitative real-time PCR (also known as RT-PCR, RQ-PCR, QRT-PCR or RTQ-PCR). In addition, many techniques exist that give estimates of relative copy numbers, as calculated relative to a reference. Such techniques include many array-based techniques. Absolute copy number estimates may be obtained by in situ hybridization techniques such as, for example, fluorescence in situ hybridization or chromogenic in situ hybridization.

Fluorescence in situ hybridization permits the analysis of copy numbers of individual genomic locations and can be used to study copy numbers of individual genetic loci or particular regions on a chromosome (see, e.g., Pinkel et al., Proc. Natl. Acad. Sci. U.S.A. (1988), 85, 9138-42). Comparative genomic hybridization can also be used to probe for copy number changes of chromosomal regions (see, e.g., Kallioniemi et al., Science (1992), 258: 818-21; and Houldsworth et al., Am. J. Pathol. (1994), 145: 1253-60).

Copy numbers of genomic locations may also be determined using quantitative PCR techniques such as real-time PCR (see, e.g., Suzuki et al., Cancer Res. (2000), 60:5405-9). For example, quantitative microsatellite analysis can be performed for rapid measurement of relative DNA sequence copy numbers. In quantitative microsatellite analysis, the copy numbers of a test sample relative to a reference sample is assessed using quantitative, real-time PCR amplification of loci carrying simple sequence repeats. Simple sequence repeats are used because of the large numbers that have been precisely mapped in numerous organisms. Exemplary protocols for quantitative PCR are provided in Innis et al., PCR Protocols, A Guide to Methods and Applications (1990), Academic Press, Inc. N.Y. Semi-quantitative techniques that may be used to determine specific DNA copy numbers include, for example, multiplex ligation-dependent probe amplification (see, e.g., Schouten et al. Nucleic Acids Res. (2002), 30(12):e57; and Sellner et al., Human Mutation (2004), 23(5):413-419) and multiplex amplification and probe hybridization (see, e.g., Sellner et al. (2004), supra).

### BRCA1 Array Comparative Genomic Hybridization

In various aspects, the present disclosure relates to the use of a BRCA1 aCGH classifier capable of identifying BRCA1-associated tumors in predicting an individual subject's response to anti-cancer therapy. In various aspects, a BRCA1 aCGH classifier capable of identifying BRCA1-associated tumors is set forth on a BRCA1 array as described herein.

Using the methods described above, in various aspects, a BRCA1 aCGH classifier is capable of detecting genomic copy number variations in a test sample, as compared to a reference sample, in at least one, or a plurality, of the genomic loci selected from 1p35-21, 3q22-27, 5p13, 5q21-34, 6p25-22, 7p21-15, 7q31-36, 8q22-24, 10p15-14, 10p12, 12p13, 12q21-23, 13q31-33, 14q22-24, 15q14-21 and 21q11-22. Using the methods described above, in various aspects, a BRCA1 aCGH classifier is capable of detecting genomic copy number variations in a test sample, as compared to a reference sample, in at least one, or a plurality, of the genomic loci selected from 1p35.1-21.3, 3q22.2-27.2, 5p13.2, 5q21.3-34, 6p25.2-22.1, 7p21.3-15.3, 7q31.33-36.3, 8q22.1-24.3, 10p15.3-14, 10p12.1, 12p13.33-13.2, 12q21.2-23.3, 13q31.2-33.3, 14q22.1-24.1, 15q14-21.1 and 21q11.2-22.3. Using the methods described above, in various aspects, a BRCA1 aCGH classifier is capable of detecting genomic copy number variations in a test sample, as compared to a reference sample, in at least one, or a plurality, of the genomic loci set forth in FIG. 1.

Using the methods described above, in various aspects, a BRCA1 aCGH classifier is capable of detecting genomic copy number variations in a test sample, using at least one, or a plurality, of probes that independently hybridize to at least one genomic locus set forth in FIG. 1. Using the methods described above, in various aspects, a BRCA1 aCGH classifier is capable of detecting genomic copy number variations in a test sample, as compared to a reference sample, using at least one, or a plurality, of the distinct BAC clones set forth in FIG. 2.

### Therapeutic Uses

In various aspects, the BRCA1 classifiers can be used to predict an individual subject's response to anti-cancer therapy.

Using the methods described above, in various aspects, the BRCA1 classifiers are capable of determining whether an individual metastatic breast cancer patient, in continuous complete remission after high dose alkylating chemotherapy, has a BRCA1-associated tumor. Using the methods described above, in various aspects, the BRCA1 classifiers are capable of determining whether a metastatic breast cancer patient with a BRCA1-associated tumor has a significantly higher complete remission rate. The BRCA1 classifiers are therefore capable of predicting response to anti-cancer therapy in an individual patient. Using the methods described above, in various aspects, the BRCA1 classifiers are capable of predicting improved outcome after platinum-based high dose alkylating chemotherapy by identifying breast cancer patients specifically benefiting from HD-chemotherapy within ER-low and HER2-negative stage-III breast cancer.

The BRCA1 classifiers can be used as pre-selection tools, to prospectively detect subjects with a high risk of carrying a BRCA1-mutation and/or a BRCA1-associated tumor. Additionally, the BRCA1 classifiers can be used as predictive tests to identify breast cancer patients likely to benefit from anti-cancer therapy.

The BRCA1 classifiers can also be used to detect a BRCA1 profile in ER+ luminal sporadic tumors. It is therefore believed that the BRCA1 classifiers and the second series BRCA1 classifiers can also be used as predictive tests to identify breast cancer patients having ER+ luminal sporadic tumors who are likely to benefit from anti-cancer therapy.

For the first time, in this disclosure clinical evidence has been provided to show that patients with so-called “triple negative” sporadic tumors who also display a BRCA1 profile, as determined by the BRCA1 classifiers, are more sensitive and respond better to high dose alkylating chemotherapy containing carboplatin, thiotepa, and cyclophosphamide (see the following Examples). Therefore, the use of the BRCA1 classifiers can be used to prospectively predict how an individual subject will respond to anti-cancer therapy. Until the present disclosure, no such test had been available.

As shown in the following Examples, the BRCA1 classifiers have been applied, via aCGH, to search for “BRCA1-like” patterns in metastatic tumors. Those patterns, where found, have been related to the treatment results of anti-cancer therapy. What was discovered was that all of the long-term survivors of stage 1V breast cancer had tumors that displayed the BRCA1-like patterns discoverable by the BRCA1 classifiers. It is also shown that triple-negative tumors that displayed the BRCA1-like patterns benefited markedly from high-dose alkylating therapy in the adjuvant setting, while the triple-negative tumors displaying sporadic-like patterns did not.

The examples provide evidence of a relation between the BRCA1-like pattern, detectable by the BRCA1 classifiers, and better treatment response to anti-cancer therapy. The examples also provide evidence that BRCA1 inactivation in triple negative tumors, which can be obtained by the use of the BRCA1 classifiers, may identify patients that respond better to alkylating agents.

The BRCA1 classifiers can be used in a clinical setting to detect the presence or absence of homologous recombination deficiency in ER-low, HER2-negative stage-III breast cancer patients. The examples disclose a comparison of the rates of cancer recurrence in patients treated according to the BRCA1-classifiers (i.e. patients with a BRCA1-like tumor: HD-chemotherapy, others: conventional chemotherapy) with the rates of cancer recurrence in patients treated with conventional chemotherapy (substitute of current clinical practice) resulted in a multivariate HR of 0.47 (95% CI 0.23-0.91). As shown in the Examples, recurrence rates for ER-low, HER2-negative stage-III breast cancers can be cut in half by utilizing the BRCA1 classifiers to tailor chemotherapy treatment.

In further aspects, the present disclosure relates to kits for use in the diagnostic applications described above. The kits can comprise any or all of the reagents to perform the methods described herein. The kits can comprise one or more of the BRCA1 classifiers. In the diagnostic applications such kits may include any or all of the following: assay reagents, buffers, nucleic acids such hybridization probes and/or primers that specifically bind to at least one of the genomic locations described herein, as well as arrays comprising such nucleic acids. In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples describe in detail the therapeutic efficacy of chemotherapy by detecting phenotypic genetic traits using comparative genomic hybridization. It will be apparent to those skilled in the art that many modifications, both to materials and methods, may be practiced without departing from the scope of the disclosure.

### Example 1

The present inventors have developed a BRCA1-classifier (FIG. 2) to identify tumors of metastatic breast cancer (MBC) patients (n=39) with a long progression-free survival after treatment with high dose platinum-based alkylating chemotherapy (HD-chemotherapy). This classifier was prospectively validated in estrogen-receptor low, HER2-negative tumors of stage-III breast cancer patients (n=77), who had been randomized between adjuvant HD-chemotherapy and conventional chemotherapy. Additionally, the concordance between the BRCA1-classifier and BRCA1-mutations in the MBC tumors was assessed.

The new classifier scored 16/39 tumors as BRCA1-like in the MBC-series (of which 2 harbored a BRCA1-mutation). In the adjuvant validation series, patients with BRCA1-like tumors (39/77=51%) benefited more from HD-chemotherapy than those with Sporadic-like tumors (38/77=49%) (test for interaction p=0.026). HD-chemotherapy strongly decreased the risk of recurrence (HR=0.15, p=0.001; 5-year recurrence free survival (RFS) 78% versus 29%), while RFS in the Sporadic-like group was not improved by HD-chemotherapy.

Based on these results, it is apparent that the benefit of intensive alkylator-based chemotherapy for the treatment of BRCA1-like tumors may outweigh the side-effects of this regimen. Furthermore, this BRCA1-classifier may represent an effective test to identify BRCAness in breast cancers and may therefore predict effectiveness of other HRD-targeting agents such as poly(ADP-ribose)polymerase(PARP)-inhibitors.

It has been suggested that Comparative Genomic Hybridization (CGH) can be useful in identifying the genomic instability inherent to HRD tumors by visualizing the copy number aberrations (CNAs)⁸. In the Netherlands Cancer Institute, a conditional knockout mouse model for BRCA1 breast tumors has been generated¹⁸. Using this model, mouse mammary tumors lacking BRCA1 were shown to be extremely sensitive to cisplatin⁷. Furthermore, these tumors displayed striking genomic instability measured by the extent of CNAs using CGH¹⁸. These findings support the use of this model to discern tumors with HRD as has been suggested by Turner et al⁸. For this study, a BRCA1 CGH classifier, designed to identify human BRCA1-mutated breast cancers from sporadic breast cancers was constructed^{19;20}. This classifier was translated to an array based platform (aCGH) and consisted of the characteristic CNAs of breast cancers from a patient series of known BRCA1 germ-line carriers.

For purposes of this study, it was hypothesized that these characteristic CNAs would not only be present in tumors with a BRCA1-mutation, but also in tumors with a wider range of molecular defects in the BRCA1-pathway. If true, this BRCA1-classifier would be capable of predicting sensitivity to DSB-inducing agents, such as alkylating agents and the new PARP-inhibitors, in breast cancer patients. To test this hypothesis, patients were studied who had been treated with one of the few regimens in which only alkylating agents were used: high dose platinum-based alkylating chemotherapy (HD-chemotherapy). It was demonstrated that this classifier was capable of selectively predicting improved outcome after HD-chemotherapy in estrogen receptor (ER)-low, HER2-negative stage III breast cancer patients who participated in a randomized trial of adjuvant HD-chemotherapy versus conventional chemotherapy.

### Methods

To determine whether the BRCA1-classifier predicts benefit from HD-chemotherapy, two patient series were studied. First, patients with metastatic breast cancer (MBC) who had received HD-chemotherapy (5-fluorouracil, epirubicin, cyclophosphamide (FEC) as induction followed by high dose cyclophosphamide, thiotepa and carboplatin (CTC) with autologous stem cell support) were studied. Since the aim of this study was different from the aim for which the classifier was initially developed, a new cut-off of the BRCA1-probability score of the BRCA1-classifier in this patient series was determined. To validate the cut-off and determine whether the BRCA1-classifier was a predictive marker, stage III breast cancer patients were studied in the adjuvant setting who had been randomized to either conventional or HD-chemotherapy (CTC) with autologous stem cell support. All trials described herein were approved by the Institutional Review board of the Netherlands Cancer Institute. This study was designed following the REMARK guidelines (Appendix 1)²².

### Patient Selection First Series (MBC Series)

Patients were included from three pilot studies carried out at the Netherlands Cancer Institute between 1993 and 2004 (one patient was included in 1989 with the setup of the trial)²³⁻²⁶. Inclusion criteria have been published previously²³⁻²⁵.

Patients were eligible when their formalin-fixed paraffin-embedded (FFPE) primary tumor tissue contained more than 60% of tumor cells and when they had received at least one course of CTC. Exclusion criteria consisted of progressive disease on induction chemotherapy (FEC), as these patients did not proceed to HD-chemotherapy; treatment-related death; contralateral breast cancer; stage IIIc²⁶ breast cancer.

### Patient Selection Second Series (Stage-III Series)

Patients of the second series were selected from a large randomized controlled multicentre trial performed in the Netherlands between 1993 and 1999. Inclusion criteria have been published previously²⁷. Eligible patients were randomized between either conventional chemotherapy (five courses FEC), or HD-chemotherapy which was identical except that instead of the fifth course of FEC, a course of CTC was given. Based on previous experience that BRCA1-like tumors virtually always have a low ER and negative HER2 expression and comprise about 30-50% of all ER-low, HER2-negative tumors, patients with tumors with a low ER expression (<25%) and a HER2-negative status in this randomized trial were studied. Cases were only included when their FFPE primary tumor tissue was available and contained more than 60% of tumor cells.

### Comparative Genomic Hybridization and Mutation Analyses

Genomic DNA was extracted from all FFPE primary tumors as previously described²⁸. Of seven patients only lymph node tissue, removed at first diagnosis containing primary tumor tissue, was available. Tumor DNA and reference DNA were labeled and hybridized as published previously and as disclosed herein²⁹. The data discussed in this Example have been deposited in NCBI's Gene Expression Omnibus and are accessible through GEO Series accession number GSE12127.

A BRCA1-classifier (FIG. 2) was constructed and refined for two purposes; 1) to use as a pre-selection tool to detect subjects with a high risk of carrying a BRCA1-mutation, which resulted in a slightly modified version³⁰; and 2) to use as a predictive test to identify breast cancer patients likely to benefit from DSB-inducing agents. For the latter, the original classifier was used as described herein. BRCA1 class detection was performed on each individual aCGH tumor profile using the BRCA1-classifier (FIG. 2), resulting in a BRCA1-probability score ranging from 0 to 1. All protocols used for aCGH are described in FIG. 3.

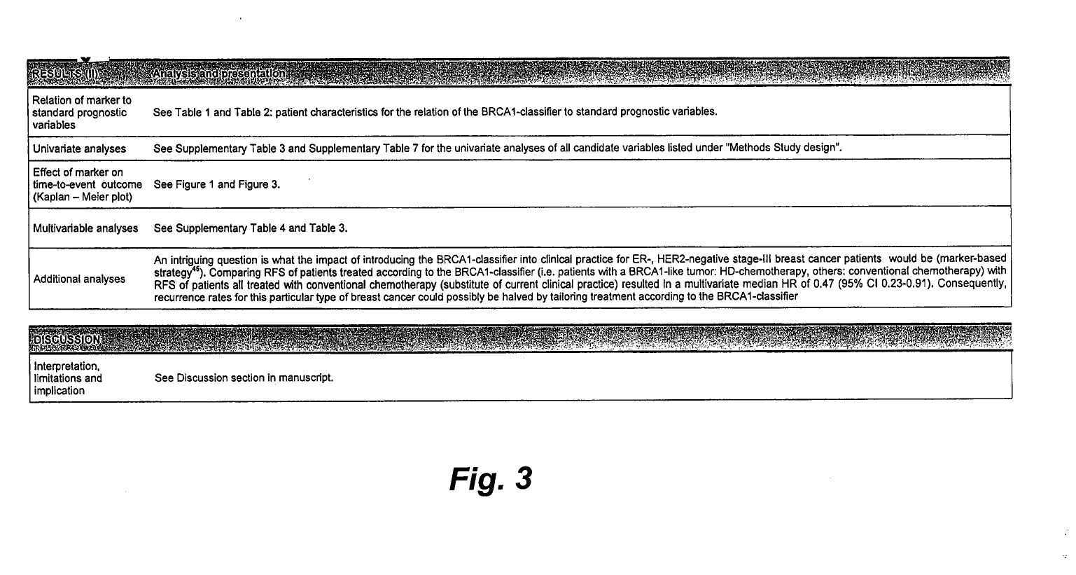

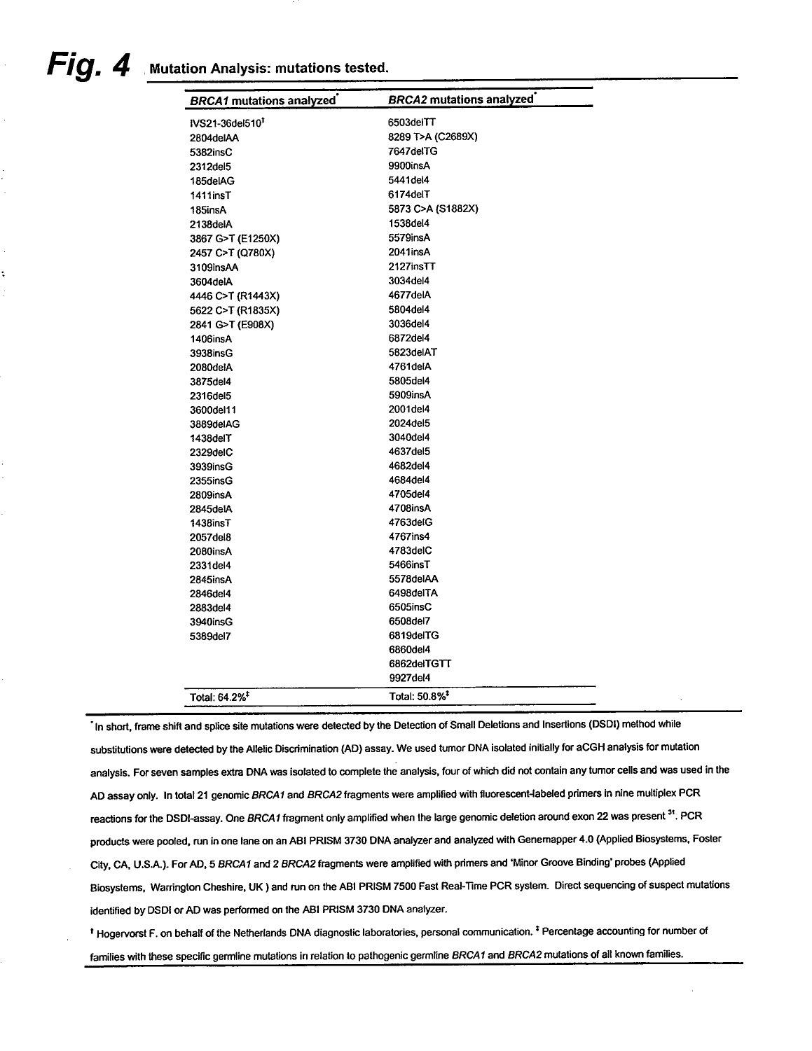

For mutation analysis a method developed especially for DNA isolated from FFPE material was utilized. The most common mutations reported in Dutch families known to carry pathogenic germline BRCA1 or BRCA2 mutations were screened. The analysis included 37 distinct BRCA1 mutations accounting for 749 of 1166 BRCA1 families (˜64%) and 40 distinct BRCA2 mutations accounting for 264 of 520 BRCA2 families (˜51%) in the Netherlands (FIG. 4).

### Histopathology

Two pathologists reviewed all tumors and scored whole H&E-slides for tumor percentages. ER, HER2 and progesterone receptor status was determined by immunohistochemistry (IHC) as described before^{27;32}. Pronase was used as pretreatment for EGFR (EGFR Ab-10 clone 111.6; 1:200; Neomarkers; EGFR clone 31 G7, 1:400; Zymed) and the standard procedure for CK 5/6 (clone D5/16 B4, M7237, 1:200, Dako). CK5 and EGFR were considered positive if any (weak or strong) staining of tumor cells was observed. Tumors were classified as basal-like according to the Nielsen basal-like breast cancer IHC definition, as published previously³³.

### Statistical Analysis

The cut-off of the BRCA1-probability score on the MBC series was determined to obtain the highest positive predictive value for response (defined as a progression free survival (PFS) longer than 24 months, the median overall survival of MBC patients) and validated in the stage-III series.

Differences between groups of interest were tested using Fisher's exact tests and exact Chi-square test for trend. Patients with missing values for a variable were excluded from analyses involving that variable. Survival curves were generated using the Kaplan-Meier method and compared using the log-rank test. Hazard ratios (HR) were calculated using Cox proportional hazards regression.

In the MBC series, complete remission after CTC-treatment was defined as disappearance of all evaluable tumor mass assessed by physical examination and imaging studies. PFS was defined as the time from the first CTC-course to the appearance of the first progression of disease (based on clinical signs and symptoms, substantiated with imaging and/or biochemical analyses and/or cytology/histology), or death, whichever occurred earlier. Patients who did not experience a progression were censored at the end of follow-up. Because of the small sample size, potential confounders were not added at once but one at a time to a model including the BRCA1-classifier.

In the stage-III series, recurrence free survival (RFS) was calculated from randomization to the appearance of a local or regional recurrence, metastases or to death from any cause²⁷. All other events were censored. Overall survival (OS) was time from randomization to death from any cause, or end of follow-up. Patients alive at their last follow-up visit at the time of analysis were censored at that time. All treatment comparisons were based on patients who completed their assigned treatment (per-protocol analysis). The effect of HD-chemotherapy versus conventional chemotherapy on RFS was assessed, expressed as hazard ratio (HR), differed by BRCA1-like status based on multivariate proportional hazards regression with an interaction term, adjusting for potential confounders.

All calculations were performed using the statistical package SPSS 15.0 and SAS 9.1 (for Windows, respectively SAS Institute Inc., Cary, N.C., USA).

### Results

### MBC Series

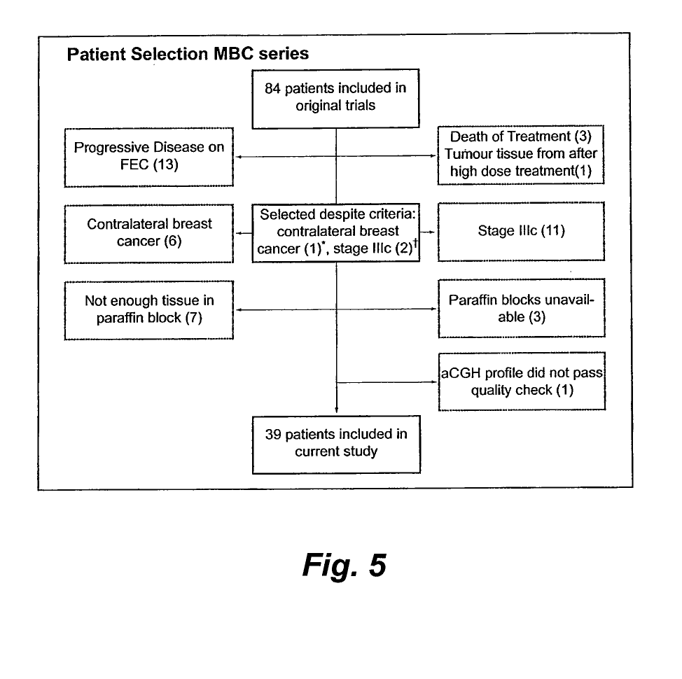

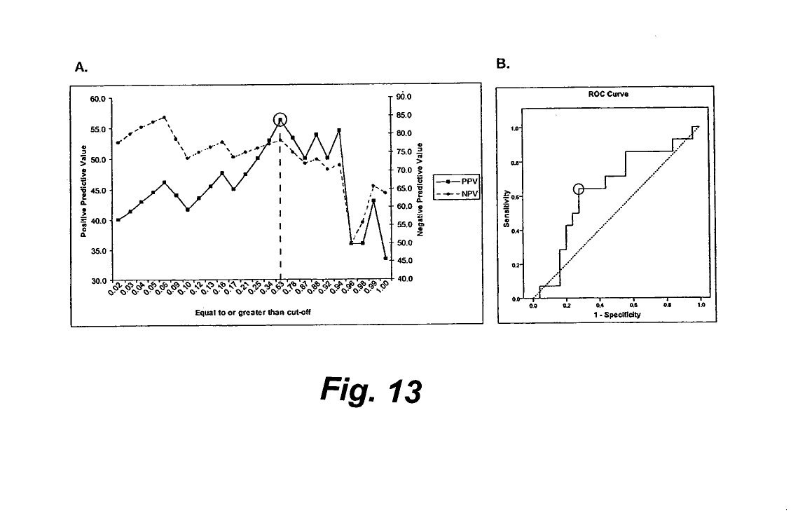

Based on aCGH-profiles of 39 patients (FIG. 5), tumors with a BRCA1-probability score >0.63 (FIG. 13) were considered to be BRCA1-like (N=16, 41%) and others as Sporadic-like (N=23, 59%). Compared with Sporadic-like tumors, BRCA1-like tumors were more often HER2-receptor negative (p=0.06), ER-negative (p=0.02), and basal-like (p<0.001) (Table 1).

TABLE 1
Patient characteristics by profile of the MBC-series

| | Patients with Sporadic-like | | Patients with BRCA1-like | | |
|---|---|---|---|---|---|
| | tumors | | tumors | | |
| Variable | n | % | n | % | p-value |
| Total | 23 | 100 | 16 | 100 | |
| Age at CTC* | | | | | |
| Mean (years) | 46.5 | | 40.0 | | 0.122 |
| Range (years) | 23.0-59.5 | | 32.6-51.0 | | |
| ≦40 years | 7 | 30.4 | 8 | 50.0 | 0.318 |
| >40 years | 16 | 69.6 | 8 | 50.0 | |
| Metastatic disease* | | | | | |
| ≦2 sites of metastases | 12 | 52.2 | 10 | 62.5 | 0.743 |
| >2 sites of metastases | 11 | 47.8 | 6 | 37.5 | |
| Histological grade^{†} | | | | | |
| Grade 1 and 2 | 9 | 39.1 | 4 | 25.0 | 0.495 |
| Grade 3 | 14 | 60.9 | 12 | 75.0 | |
| HER2 receptor^{†} | | | | | |
| Negative | 15 | 65.2 | 15 | 93.8 | 0.056 |
| Positive | 8 | 34.8 | 1 | 6.3 | |
| Estrogen receptor status^{†} | | | | | |
| Negative | 11 | 47.8 | 14 | 87.5 | 0.017 |
| Positive | 12 | 52.2 | 2 | 12.5 | |
| Progesterone receptor status^{†} | | | | | |
| Negative | 11 | 47.8 | 12 | 75.0 | 0.240 |
| Positive | 6 | 26.1 | 2 | 12.5 | |
| Unknown | 6 | 26.1 | 2 | 12.5 | |
| CK 5/6 status^{†} | | | | | |
| Negative | 22 | 95.7 | 8 | 50.0 | 0.001 |
| Positive | 1 | 4.3 | 8 | 50.0 | |
| EGFR status^{†} | | | | | |
| Negative | 19 | 82.6 | 9 | 56.2 | 0.024 |
| Positive | 2 | 8.7 | 7 | 43.8 | |
| Unknown | 2 | 8.7 | 0 | 0.0 | |
| Nielsen basal-like breast cancer | | | | | |
| definition^{†} | | | | | |
| Negative | 22 | 95.7 | 7 | 43.8 | <0.001 |
| Positive | 1 | 4.3 | 9 | 56.2 | |
| Prior Chemotherapy^{‡} | | | | | |
| No | 13 | 56.5 | 14 | 87.5 | 0.076 |
| Yes | 10 | 43.5 | 2 | 12.5 | |
| Prior Radiotherapy | | | | | |
| No | 5 | 21.7 | 5 | 31.3 | 0.711 |
| Yes | 18 | 78.3 | 11 | 68.8 | |
| Number of CTC courses | | | | | |
| <3 courses | 9 | 39.1 | 3 | 18.8 | 0.291 |
| 3 courses | 14 | 60.9 | 13 | 81.3 | |
| CTC Response | | | | | |
| All other responses | 14 | 60.9 | 3 | 18.8 | 0.020 |
| Complete Remission | 9 | 39.1 | 13 | 81.3 | |

*at start first CTC treatment.
†Of primary tumor, except for two patients of whom only the lymph node metastasis tissue of the primary tumor was available.
‡Prior chemotherapy, in all cases consisted of cyclophosphamide, methotrexate and fluoruracil (CMF) in the adjuvant setting, except one case who received five courses of adjuvant FE₉₀C.
Missing data excluded from analysis; p-value calculated using the Fisher exact test. Abbreviations: CI, confidence interval; IHC, immunohistochemistry; CTC, carboplatin-thiotepa-cyclophosphamide.

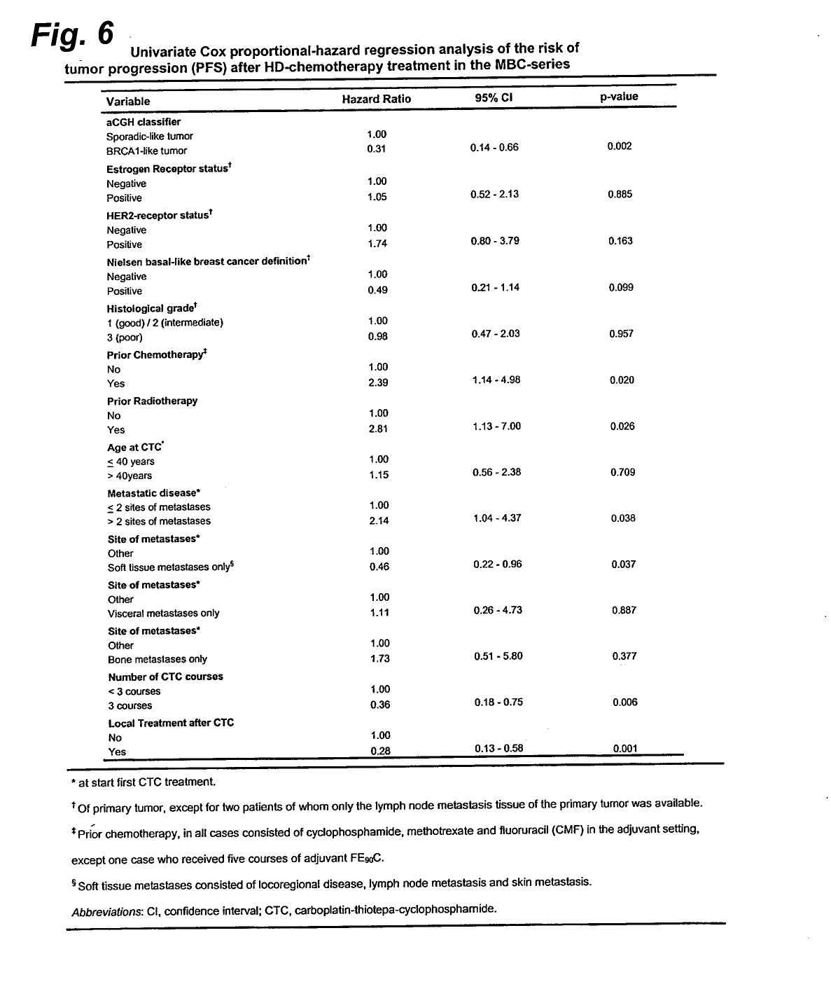

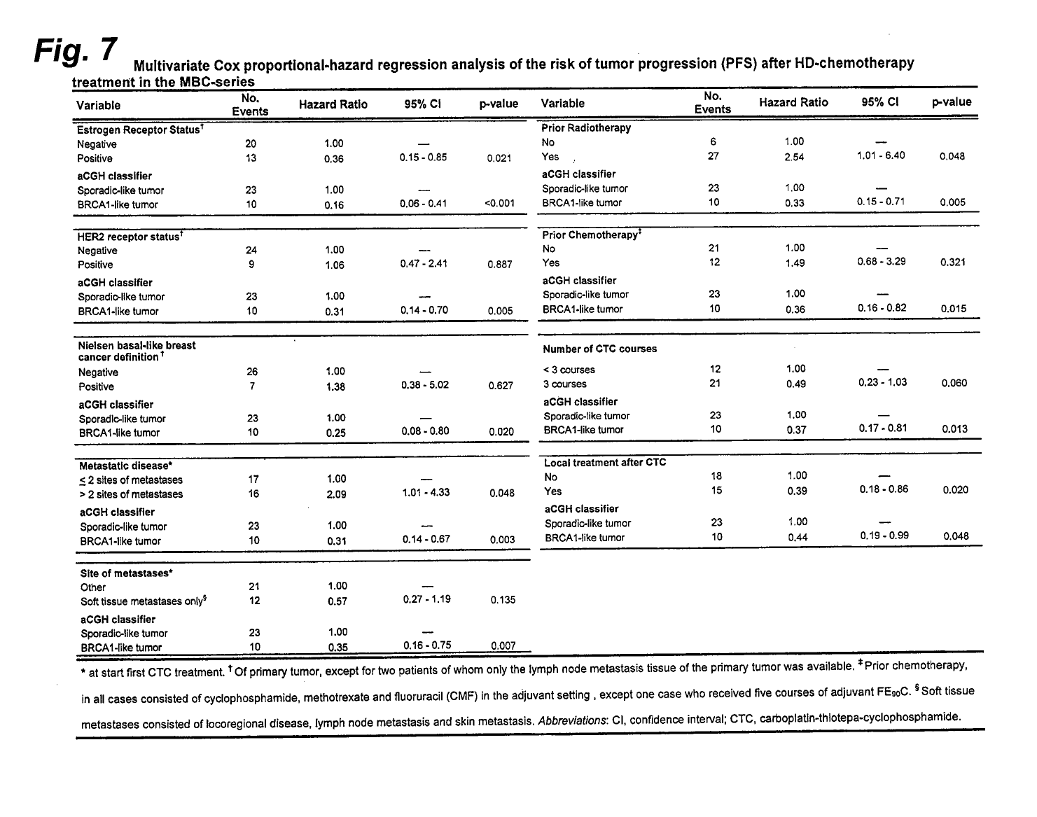

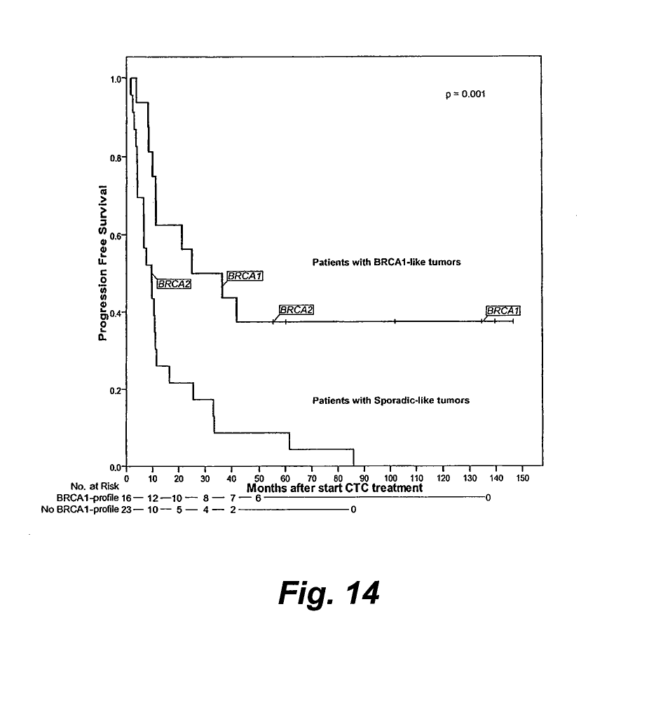

BRCA1-like patients had a significantly better response to CTC-treatment, defined by achievement of complete remission (p=0.02), and significantly longer PFS (FIG. 14, p=0.001), with a univariate HR for progression of 0.31 (95% CI: 0.14-0.66, FIG. 6). Adjustment for potential confounders did not substantially modify the HR (FIG. 7).

### MBC Series and Mutation Analysis

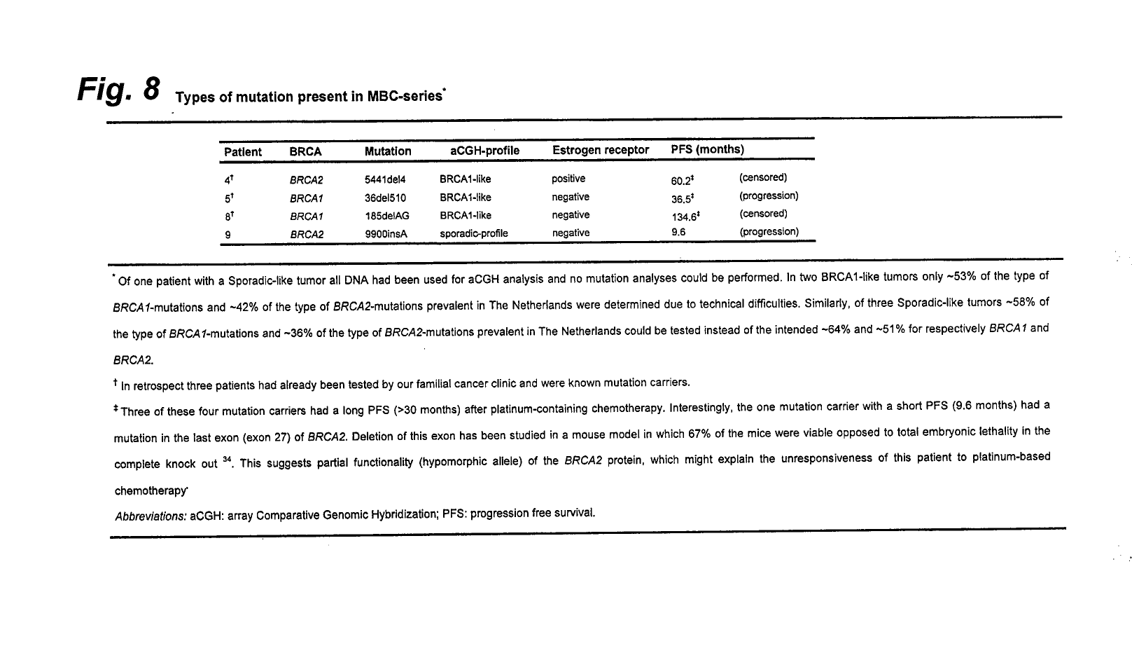

Two BRCA1-mutated tumors, both of which had a BRCA1-like tumor were identified. Additionally, two BRCA2-mutated tumors were identified, one of which had a BRCA1-like tumor (FIG. 8). Mutations were not necessarily germ-line mutations since DNA derived from the tumors was tested. In fact, three of the four BRCA-mutated patients identified in this analysis had been tested by a familial cancer clinic and were known mutation carriers. The familial cancer clinic had tested one additional patient of this study, who was found to be wild type BRCA1/2 in both analyses. For one patient, all DNA was used for aCGH, and mutation analyses could not be performed.

### Stage-III Series

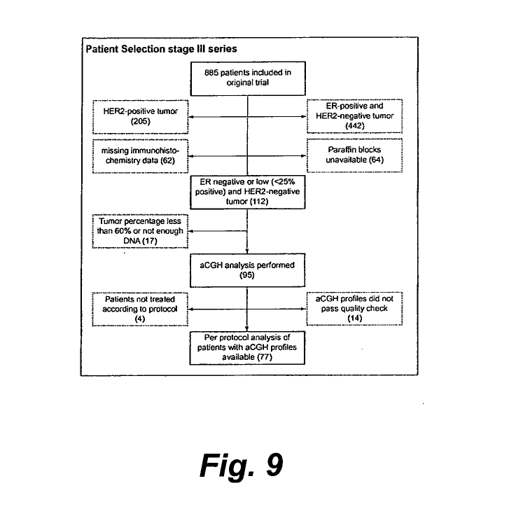

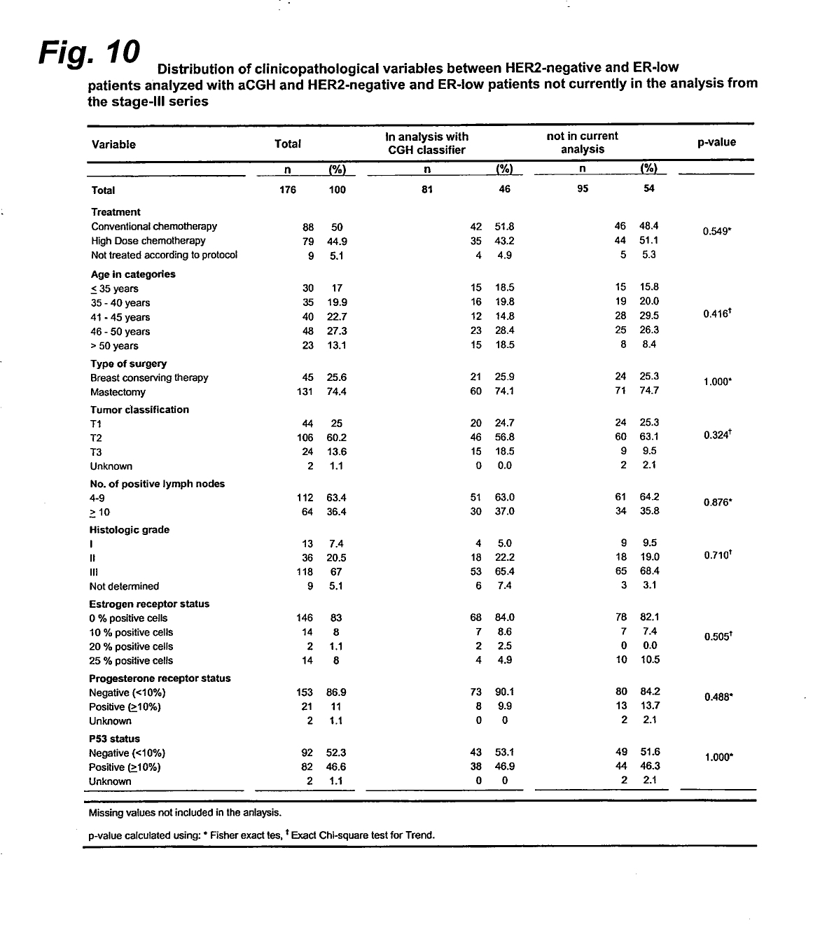

FIG. 9 summarizes the flow of patients through the study including the number of patients in each stage. Reasons for dropout are listed. Tumor aCGH profiles could be obtained for 81 patients. Characteristics and treatments of these 81 patients did not differ from those of the ER-low, HER2-negative patients not in the current analysis (FIG. 10). Four of these 81 patients were not treated according to protocol and were excluded from further analysis.

Of the 77 patients, 39 tumors (51%) were scored as BRCA1-like. Patient characteristics did not differ by treatment arm within the patients with BRCA1- or Sporadic-like tumors (Table 2). Patients with BRCA1-like tumors were generally younger, and their tumors were more often poorly differentiated and progesterone receptor negative. Tumor size according to TNM classification, number of positive lymph nodes and treatment were significantly associated with RFS (FIG. 11) and therefore included in multivariate analyses as potential confounders.

TABLE 2
Patient characteristics distributed by treatment arm per BRCA1-classification of the stage-III series

| | | | Patients with Sporadic-like tumors | | | | | Patients with BRCA1-like tumors | | | | |
|---|---|---|---|---|---|---|---|---|---|---|---|---|
| | | | Conventional | | High Dose | | | Conventional | | High Dose | | |
| | Total | | Chemotherapy | | chemotherapy | | p- | Chemotherapy | | chemotherapy | | p- |
| Variable | n | % | n | % | n | % | val [symbol] | n | % | n | % | val [symbol] |
| Total | 77 | 1 [symbol] | 21 | 53.8 | 17 | 44.7 | | 21 | 53.8 | 18 | 46.2 | |
| Age in [symbol] | | | | | | | | | | | | |
| ≦35 years | 14 | 1 [symbol] | 3 | 14.3 | 2 | 11.8 | 0.820^{†} | 5 | 23.8 | 4 | 22.2 | 0.715^{†} |
| 35-40 years | 16 | 2 [symbol] | 2 | 9.5 | 4 | 23.5 | | 5 | 23.8 | 5 | 27.8 | |
| 41-45 years | 11 | 1 [symbol] | 2 | 9.5 | 2 | 11.8 | | 4 | 19.0 | 3 | 16.7 | |
| 46-50 years | 22 | 2 [symbol] | 9 | 42.9 | 3 | 17.6 | | 7 | 33.3 | 3 | 16.7 | |
| >50 years | 14 | 1 [symbol] | 5 | 23.8 | 6 | 35.3 | | 0 | 0.0 | 3 | 16.7 | |
| Type of | | | | | | | | | | | | |
| Mastectomy | 57 | 7 [symbol] | 17 | 81.0 | 14 | 82.4 | 1.000* | 14 | 66.7 | 12 | 66.7 | 1.000* |
| Breast | 20 | 2 [symbol] | 4 | 19.0 | 3 | 17.6 | | 7 | 33.3 | 6 | 33.3 | |
| Tumor [symbol] | | | | | | | | | | | | |
| T1 | 17 | 2 [symbol] | 2 | 9.5 | 3 | 17.6 | 0.605^{†} | 6 | 28.6 | 6 | 33.3 | 0.458^{†} |
| T2 | 45 | 5 [symbol] | 13 | 61.9 | 10 | 58.8 | | 11 | 52.4 | 11 | 61.1 | |
| T3 | 15 | 1 [symbol] | 6 | 28.6 | 4 | 23.5 | | 4 | 19.0 | 1 | 5.6 | |
| No. of positive [symbol] | | | | | | | | | | | | |
| 4-9 | 48 | 6 [symbol] | 14 | 66.7 | 9 | 52.9 | 0.509* | 14 | 66.7 | 11 | 61.1 | 0.750* |
| ≧10 | 29 | 3 [symbol] | 7 | 33.3 | 8 | 47.1 | | 7 | 33.3 | 7 | 38.9 | |
| Histologic [symbol] | | | | | | | | | | | | |
| I | 4 | 5. [symbol] | 1 | 4.8 | 3 | 17.6 | 1.000^{†} | 0 | 0.0 | 0 | 0.0 | 0.626* |
| II | 16 | 2 [symbol] | 9 | 42.9 | 3 | 17.6 | | 3 | 14.3 | 1 | 5.6 | |
| III | 51 | 6 [symbol] | 9 | 42.9 | 10 | 58.8 | | 18 | 85.7 | 14 | 77.8 | |
| Not determined | 6 | 7. [symbol] | 2 | 9.5 | 1 | 5.9 | | 0 | 0.0 | 3 | 16.7 | |
| Estrogen | | | | | | | | | | | | |
| 0% positive [symbol] | 65 | 8 [symbol] | 14 | 66.7 | 15 | 88.2 | 0.249^{†} | 19 | 90.5 | 17 | 94.4 | 1.000^{†} |
| 10% positive [symbol] | 6 | 7. [symbol] | 4 | 19.0 | 1 | 5.9 | | 1 | 4.8 | 0 | 0.0 | |
| 20% positive [symbol] | 2 | 2. [symbol] | 1 | 4.8 | 0 | 0.0 | | 1 | 4.8 | 0 | 0.0 | |
| 25% positive [symbol] | 4 | 5. [symbol] | 2 | 9.5 | 1 | 5.9 | | 0 | 0.0 | 1 | 5.6 | |
| Progesterone | | | | | | | | | | | | |
| Negative [symbol] | 69 | 8 [symbol] | 16 | 76.2 | 15 | 88.2 | 0.427* | 21 | 100.0 | 17 | 94.4 | 0.462* |
| Positive (≧10%) | 8 | 1 [symbol] | 5 | 23.8 | 2 | 11.8 | | 0 | 0.0 | 1 | 5.6 | |
| P53 status | | | | | | | | | | | | |
| Negative [symbol] | 43 | 5 [symbol] | 11 | 52.4 | 11 | 64.7 | 0.521* | 11 | 52.4 | 10 | 55.6 | 1.000* |
| Positive (≧10%) | 34 | 4 | 10 | 47.6 | 6 | 35.3 | | 10 | 47.6 | 8 | 44.4 | |

Missing values not included in the statistical analyses. p-value calculated using:
* Fisher exact test;
^{†}Exact Chi-square test for Trend. [symbol]
indicates data missing or illegible when filed

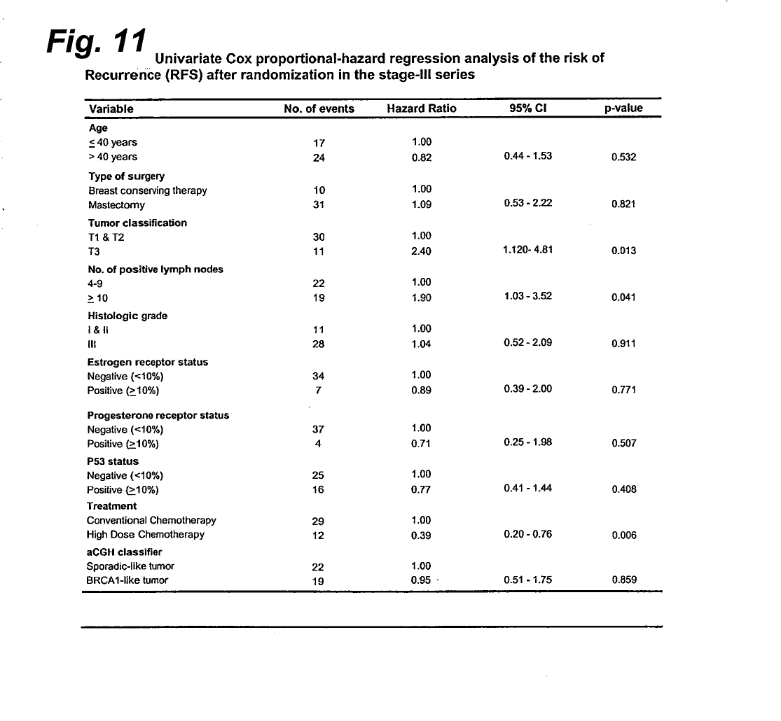

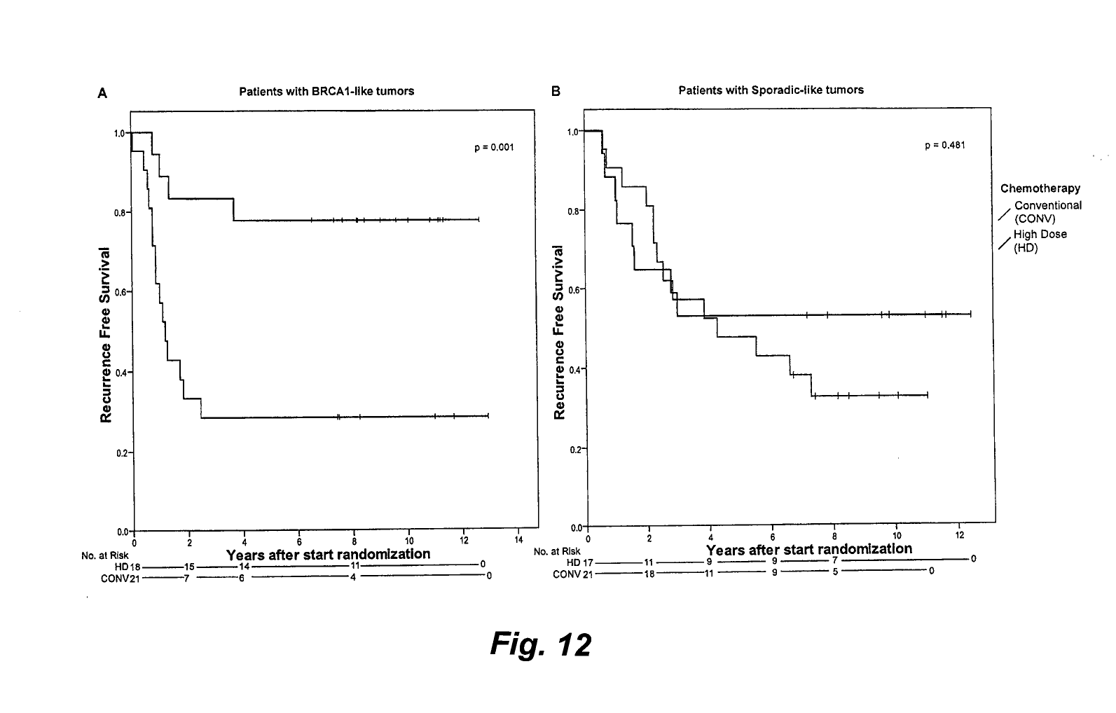

The beneficial effect of HD-chemotherapy differed significantly between patients with BRCA1-like tumors and those with Sporadic-like ones (test for interaction p=0.03). Among patients with BRCA1-like tumors, the risk of recurrence was almost 7-fold decreased after HD-chemotherapy compared to conventional chemotherapy (multivariate HR 0.15, 95% CI 0.05-0.46, p=0.001, FIG. 12, Table 3), while in patients with Sporadic-like tumors no significant difference was observed (multivariate HR 0.74, 95% CI 0.31-1.77, p=0.50, FIG. 12, Table 3).

TABLE 3
Multivariate Cox proportional-hazard analysis of the risk of
recurrence (RFS) in the stage-III series

| Variable | No. Events | Hazard Ratio | 95% CI | p-value |
|---|---|---|---|---|
| Lymph Nodes | | | | |
| 4-9 LN positive | 22 | 1.00 | | |
| ≧10 LN positive | 19 | 2.14 | 1.11-4.13 | 0.023 |
| p T-stage | | | | |
| 1 or 2 | 30 | 1.00 | | |
| 3 | 11 | 1.94 | 0.93-4.04 | 0.079 |
| aCGH classifier | | | | |
| Sporadic-like tumor | 22 | 1.00 | | |
| BRCA1-like tumor | 19 | 2.27 | 1.06-4.88 | 0.035 |
| BRCA1-like tumor | | | | |
| Conventional chemotherapy | 15 | 1.00 | | |
| High Dose chemotherapy | 4 | 0.15* | 0.05-0.46 | 0.001 |
| Sporadic-like tumor | | | | |
| Conventional chemotherapy | 14 | 1.00 | | |
| High Dose chemotherapy | 8 | 0.74* | 0.31-1.77 | 0.498 |

*Homogeneity of both hazard ratios was rejected based on an interaction term with p = 0.026.

Similar trends were observed for overall survival (data not shown, test for interaction p=0.09), in which patients with BRCA1-like tumors benefited significantly from HD-chemotherapy (HR 0.22, 95% CI 0.07-0.66) while patients with Sporadic-like tumors appeared not to benefit (HR 0.75, 95% CI 0.29-1.90).

The aim of this study was to investigate whether an aCGH classifier (FIG. 2), initially constructed to identify BRCA1-mutated tumors, was capable of predicting response to DSB-inducing agents, such as high dose platinum-based alkylating chemotherapy. Remarkably, with this classification it was found that MBC patients who were in continuous complete remission (55 to 147 months) after high dose alkylating chemotherapy all had a BRCA1-like tumor. Furthermore, BRCA1-like MBC patients had a significantly higher complete remission rate suggesting this classifier was predictive of drug response. To validate the BRCA1-classifier and prove that it indeed predicted for response to HD chemotherapy, the classifier was applied to tumor DNA of stage-III breast cancer patients selected from a large trial in which patients had been randomized between conventional adjuvant chemotherapy of that time and a HD-chemotherapy regimen similar to the one used in MBC patients. It was found that the BRCA1-classifier predicted for improved outcome after platinum-based high dose alkylating chemotherapy by identifying breast cancer patients specifically benefiting from HD-chemotherapy within ER-low and HER2-negative stage-III breast cancer patients.

In the MBC series 41% (16/39) and in the stage-III series 51% (39/77) of the tumors were BRCA1-like, suggesting that the classifier identified not only BRCA1 mutation carriers but also tumors with potentially other defects in the BRCA1-pathway. To further substantiate this, mutation analysis was performed on material of the MBC series. Four patients (4/38; 11%) were identified with a mutation in BRCA1 or BRCA2 in their primary tumor. This is comparable to the reported frequency (9-12%) of BRCA1 and BRCA2 mutations in non-Dutch European breast cancer patients younger than 45 years³⁵⁻³⁷. Only three of the mutation carriers were scored as BRCA1-like (3/16, 19%), suggesting that the BRCA1-classifier also reflects other defects in the BRCA1-pathway.

A statistically significant benefit from adjuvant HD-chemotherapy with a 5-year RFS of 78% was observed in BRCA1-like patients, but not among Sporadic-like patients; this difference was statistically significant. The 5-year RFS observed in all conventionally treated stage-III patients of 38% is comparable to disease free survival rates of ER-, HER2-negative breast cancer patients treated with similar anthracycline-based regimens^{41;42}. The 5-year RFS of HD-chemotherapy remains impressive when put into perspective of current clinical practice, with 5-year disease free survival rates of 64-67% after taxane containing chemotherapy^{41;42}; especially when taking into account that those rates were observed in patients with earlier breast cancer stages than solely stage III^{41;42}.

The facts that the subgroup analysis performed was based on strong preclinical and clinical evidence of a molecular based concept (HRD and sensitivity to alkylating agents) and the information that the instant findings were confirmed in two independent datasets, provide substantial evidence for the BRCA1-classifier to be a predictive test for selective benefit of HD-chemotherapy. Moreover, one could envision that different cut-offs of the BRCA1-probability score could be used for different stages of breast cancer. For example, in metastatic patients who have exhausted their treatment options, it would be justified to set a low cut-off to ensure less false negative results (i.e. under-treatment).

Based on these results, the benefit of intensive alkylator-based chemotherapy for the treatment of BRCA1-like tumors may outweigh the side-effects of this regimen. Since response to platinum/alkylating agents is a read-out of HRD, this classifier may represent a clinical test for BRCAness in this specific subgroup. This classifier may also be predictive for other agents/regimens that target HRD, e.g. PARP-inhibitors.

### REFERENCE LIST

1. Karran P. DNA double strand break repair in mammalian cells. Curr Opin Genet Dev 2000; 10(2):144-150.
2. Khanna K K, Jackson S P. DNA double-strand breaks: signaling, repair and the cancer connection. Nat Genet 2001; 27(3):247-254.
3. van Gent D C, Hoeijmakers J H, Kanaar R. Chromosomal stability and the DNA double-stranded break connection. Nat Rev Genet 2001; 2(3):196-206.
4. Cass I, Baldwin R L, Varkey T, Moslehi R, Narod S A, Karlan B Y. Improved survival in women with BRCA-associated ovarian carcinoma. Cancer 2003; 97(9):2187-2195.
5. Garber J E, Richardson A, Harris L N et al. Neo-adjuvant cisplatin (CDDP) in “triple-negative” breast cancer (BC). Breast Cancer Res Treat 2007; (Supplement 1):S149.
6. Quinn J E, Kennedy R D, Mullan P B et al. BRCA1 functions as a differential modulator of chemotherapy-induced apoptosis. Cancer Res 2003; 63(19):6221-6228.
7. Rottenberg S, Nygren A O, Pajic M et al. Selective induction of chemotherapy resistance of mammary tumors in a conditional mouse model for hereditary breast cancer. Proc Natl Acad Sci USA 2007; 104(29):12117-12122.
8. Turner N, Tutt A, Ashworth A. Hallmarks of ‘BRCAness’ in sporadic cancers. Nat Rev Cancer 2004; 4(10):814-819.
9. Kennedy R D, Quinn J E, Mullan P B, Johnston P G, Harkin D P. The role of BRCA1 in the cellular response to chemotherapy. J Natl Cancer Inst 2004; 96(22):1659-1668.
10. Yap H Y, Salem P, Hortobagyi G N et al. Phase II study of cis-dichlorodiammineplatinum(II) in advanced breast cancer. Cancer Treat Rep 1978; 62(3):405-408.
11. Eisen T, Smith I E, Johnston S et al. Randomized phase II trial of infusional fluorouracil, epirubicin, and cyclophosphamide versus infusional fluorouracil, epirubicin, and cisplatin in patients with advanced breast cancer. J Clin Oncol 1998; 16(4):1350-1357.
12. Crown J P. The platinum agents: a role in breast cancer treatment? Semin Oncol 2001; 28(1 Suppl 3):28-37.
13. Plummer E R, Calvert H. Targeting poly(ADP-ribose) polymerase: a two-armed strategy for cancer therapy. Clin Cancer Res 2007; 13(21):6252-6256.
14. Ratnam K, Low J A. Current development of clinical inhibitors of poly(ADP-ribose) polymerase in oncology. Clin Cancer Res 2007; 13(5):1383-1388.
15. Ashworth A. A synthetic lethal therapeutic approach: poly(ADP) ribose polymerase inhibitors for the treatment of cancers deficient in DNA double-strand break repair. J Clin Oncol 2008; 26(22):3785-3790.
16. Fong P C, Boss D S, Yap T A et al. Inhibition of poly(ADP-ribose) polymerase in tumors from BRCA mutation carriers. N Engl J Med 2009; 361(2):123-134.
17. O'Shaughnessy J, Osborne C, Pippen J et al. Efficacy of BSI-201, a poly (ADP-ribose) polymerase-1 (PARP1) inhibitor, in combination with gemcitabine/carboplatin (G/C) in patients with metastatic triple-negative breast cancer (TNBC): Results of a randomized phase II trial. J Clin Oncol (Meeting Abstracts) 2009; 27(15S):3.
18. Liu X, Holstege H, van der G H et al. Somatic loss of BRCA1 and p53 in mice induces mammary tumors with features of human BRCA1-mutated basal-like breast cancer. Proc Natl Acad Sci USA 2007; 104(29):12111-12116.
19. Wessels L F, van Welsem T, Hart A A, van't Veer L J, Reinders M J, Nederlof P M. Molecular classification of breast carcinomas by comparative genomic hybridization: a specific somatic genetic profile for BRCA1 tumors. Cancer Res 2002; 62(23):7110-7117.
20. van Beers E H, van Welsem T, Wessels L F et al. Comparative genomic hybridization profiles in human BRCA1 and BRCA2 breast tumors highlight differential sets of genomic aberrations. Cancer Res 2005; 65(3):822-827.
21. Tibshirani R, Hastie T, Narasimhan B, Chu G. Diagnosis of multiple cancer types by shrunken centroids of gene expression. Proc Natl Acad Sci USA 2002; 99(10):6567-6572.
22. McShane L M, Altman D G, Sauerbrei W, Taube S E, Gion M, Clark G M. Reporting recommendations for tumor marker prognostic studies (REMARK). J Natl Cancer Inst 2005; 97(16):1180-1184.
23. Rodenhuis S, Westermann A, Holtkamp M J et al. Feasibility of multiple courses of high-dose cyclophosphamide, thiotepa, and carboplatin for breast cancer or germ cell cancer. J Clin Oncol 1996; 14(5):1473-1483.
24. Schrama J G, Baars J W, Holtkamp M J, Schornagel J H, Beijnen J H, Rodenhuis S. Phase II study of a multi-course high-dose chemotherapy regimen incorporating cyclophosphamide, thiotepa, and carboplatin in stage 1V breast cancer. Bone Marrow Transplant 2001; 28(2):173-180.
25. de Gast G C, Vyth-Dreese F A, Nooijen W et al. Reinfusion of autologous lymphocytes with granulocyte-macrophage colony-stimulating factor induces rapid recovery of CD4+ and CD8+ T cells after high-dose chemotherapy for metastatic breast cancer. J Clin Oncol 2002; 20(1):58-64.
26. Greene F, Balch C, Haller D, Morrow M. AJCC Cancer Staging Manual (6th Edition). Springer, 2002.
27. Rodenhuis S, Bontenbal M, Beex L V et al. High-dose chemotherapy with hematopoietic stem-cell rescue for high-risk breast cancer. N Engl J Med 2003; 349(1):7-16.
28. van Beers E H, Joosse S A, Ligtenberg M J et al. A multiplex PCR predictor for aCGH success of FFPE samples. Br J Cancer 2006; 94(2):333-337.
29. Joosse S A, van Beers E H, Nederlof P M. Automated array-CGH optimized for archival formalin-fixed, paraffin-embedded tumor material. BMC Cancer 2007; 7:43.
30. Joosse S A, van Beers E H, Tielen I H et al. Prediction of BRCA1-association in hereditary non-BRCA1/2 breast carcinomas with array-CGH. Breast Cancer Res Treat 2008.
31. Petrij-Bosch A, Peelen T, van Vliet M et al. BRCA1 genomic deletions are major founder mutations in Dutch breast cancer patients. Nat Genet 1997; 17(3):341-345.
32. Van De Vijver M J, Peterse J L, Mooi W J et al. Neu-protein overexpression in breast cancer. Association with comedo-type ductal carcinoma in situ and limited prognostic value in stage II breast cancer. N Engl J Med 1988; 319(19):1239-1245.
33. Nielsen T O, Hsu F D, Jensen K et al. Immunohistochemical and clinical characterization of the basal-like subtype of invasive breast carcinoma. Clin Cancer Res 2004; 10(16):5367-5374.
34. McAllister K A, Bennett L M, Houle C D et al. Cancer susceptibility of mice with a homozygous deletion in the COOH-terminal domain of the Brca2 gene. Cancer Res 2002; 62(4):990-994.
35. de Sanjose S, Leone M, Berez V et al. Prevalence of BRCA1 and BRCA2 germline mutations in young breast cancer patients: a population-based study. Int J Cancer 2003; 106(4):588-593.
36. Loman N, Johannsson O, Kristoffersson U, Olsson H, Borg A. Family history of breast and ovarian cancers and BRCA1 and BRCA2 mutations in a population-based series of early-onset breast cancer. J Natl Cancer Inst 2001; 93(16):1215-1223.
37. Peto J, Collins N, Barfoot R et al. Prevalence of BRCA1 and BRCA2 gene mutations in patients with early-onset breast cancer. J Natl Cancer Inst 1999; 91(11):943-949.
38. Esteller M, Silva J M, Dominguez G et al. Promoter hypermethylation and BRCA1 inactivation in sporadic breast and ovarian tumors. J Natl Cancer Inst 2000; 92(7):564-569.
39. Turner N C, Reis-Filho J S, Russell A M et al. BRCA1 dysfunction in sporadic basal-like breast cancer. Oncogene 2007; 26(14):2126-2132.
40. Beger C, Pierce L N, Kruger M et al. Identification of Id4 as a regulator of BRCA1 expression by using a ribozyme-library-based inverse genomics approach. Proc Natl Acad Sci USA 2001; 98(1):130-135.
41. Hayes D F, Thor A D, Dressler L G et al. HER2 and response to paclitaxel in node-positive breast cancer. N Engl J Med 2007; 357(15):1496-1506.
42. Hugh J, Hanson J, Cheang M C et al. Breast cancer subtypes and response to docetaxel in node-positive breast cancer: use of an immunohistochemical definition in the BCIRG 001 trial. J Clin Oncol 2009; 27(8):1168-1176.
43. Farquhar C M, Marjoribanks J, Lethaby A, Basser R. High dose chemotherapy for poor prognosis breast cancer: systematic review and meta-analysis. Cancer Treat Rev 2007; 33(4):325-337.
44. Rodenhuis S. The status of high-dose chemotherapy in breast cancer. Oncologist 2000; 5(5):369-375.
45. Sargent D J, Conley B A, Allegra C, Collette L. Clinical trial designs for predictive marker validation in cancer treatment trials. J Clin Oncol 2005; 23(9):2020-2027.

### Example 2

Tumors with homologous recombination deficiency (HRD), such as BRCA1 associated breast cancers, are not able to reliably repair DNA double strand breaks (DSBs), and are therefore highly sensitive to both DSB-inducing chemotherapy and PARP inhibitors. In the study presented in this Example, markers that may indicate the presence of HRD in HER2-negative breast cancers and related them to neoadjuvant chemotherapy response were studied. Array Comparative Genomic Hybridization (aCGH), BRCA1 promoter methylation, BRCA1 mRNA expression, and EMSY amplification were assessed in 163 HER2 negative pretreatment biopsies from patients scheduled for neoadjuvant chemotherapy. Features of BRCA1 dysfunction were frequent in triple-negative (TN) tumors: a BRCA1-like aCGH pattern, promoter methylation and reduced mRNA expression were observed in respectively 57%, 25% and 36% of the TN tumors. Abnormalities associated with BRCA1 inactivation are present in about half of the TN breast cancers, but were not predictive of chemotherapy response.

Neoadjuvant chemotherapy has become a widely used treatment strategy for patients with early or locally advanced breast cancer. It is equally effective as similar drug therapy following local treatment and it has additional advantages: breast conserving therapy is more frequently possible as a result of tumor shrinkage and the effect of the drugs on the tumor can be assessed during treatment. The complete disappearance of all tumor cells at microscopic examination (pathologic complete remission, or pCR) correlates well with overall survival^{[1,2]} and achieving a pCR is considered an appropriate intermediate endpoint for clinical trials. Current neoadjuvant drug regimens achieve a pCR rate of 5-10% in luminal type breast cancers, and about 40% in basal-like and in HER2/neu-positive tumors^{[3,4]}.

Bifunctional alkylators and platinating agents cause interstrand DNA crosslinking, which cause DNA double strand breaks (DSBs) during DNA replication. In normal cells, these DSBs are repaired by a process called homologous recombination. If this process is unavailable or impaired, a situation referred to as ‘homologous recombination deficiency’ (HRD) is present and alternative, error-prone DNA repair mechanisms take over, leading to genomic instability. The breast cancer genes BRCA1 and BRCA2 are essential for homologous recombination and tumors of patients carrying germ-line mutations in these genes show HRD as a result of the loss of the second, unmutated allele. BRCA1 and BRCA2 can be inactivated in sporadic cancers as well^{[5,6]}, a phenomenon referred to as ‘BRCA-ness’. Many additional genes are involved in homologous recombination, including the Fanconi anemia genes and the BRCA2 inactivating gene EMSY^{[7]}.

Tumors with HRD have been shown to be particularly sensitive to DNA crosslinking agents, such as alkylators and platinum drugs^{[8-10]}. Both classes of drugs are employed in locally advanced breast cancer. Importantly, the novel poly (ADP-ribose) polymerase (PARP)-inhibitors are specifically effective in HRD tumors as well, and have shown impressive activity in clinical studies recently^{[11-13]}. Unfortunately, no clinical tests exist which can reliably determine HRD in tumor biopsies. Previous studies have focused on genes that have a role in homologous recombination, such as the BRCA1 and -2 genes, FANC genes and EMSY^{[6]}. It has been shown that breast cancers of BRCA1 and BRCA2 mutation carriers have a characteristic pattern of DNA gains and losses in an array comparative genomic hybridization (aCGH) assay^{[5,14-18]}. In a recent study from the Netherlands Cancer Institute, a subgroup of hormone receptor negative tumors characterized by BRCA1-like aCGH pattern were shown to benefit markedly from intensive platinum-based chemotherapy^{[19]}. Another recent report showed that a subset of TN tumors might be sensitive to the DNA DSB inducing drug cisplatin, as a result of low BRCA1 expression levels or BRCA1 promoter methylation^{[20]}.

In this study, the present inventors prospectively determined the frequency in which these HRD-associated features occur in untreated patients with breast cancer. The findings were correlated with response to chemotherapy that causes DNA DSBs. If HRD is indeed confirmed to be the ‘Achilles heel’ of certain sporadic tumors, such tests could eventually serve to individualize drug treatment.

Patients

Pre-treatment biopsies of primary breast tumors from 163 women with HER2 negative breast cancer were collected. All patients had received neoadjuvant treatment at the Netherlands Cancer Institute between 2004 and 2009 as part of two ongoing clinical trials, or were treated off protocol according to the standard arm of one of these studies. Both studies had been approved by the ethical committee and informed consent was obtained from all patients. For eligibility, breast carcinoma with either a primary tumor size of at least 3 cm was required, or the presence of fine needle aspiration (FNA)-proven axillary lymph node metastases. Biopsies were taken using a 14G core needle under ultrasound guidance. After collection, specimens were snap-frozen in liquid nitrogen and stored at −70° C. Each patient had two or three biopsies taken to assure that enough tumor material was available for both diagnosis and further study.

Depending on the particular study, a treatment regimen was assigned to each patient, which consisted of one of the following: 1.) Six courses of dose-dense Doxorubicin/Cyclophosphamide (ddAC); or 2.) Six courses of Capecitabine/Docetaxel (CD); or 3.) If the therapy response was considered unfavorable by MRI evaluation after three courses, ddAC was changed to CD or vice versa. For the current study, only patients who started with ddAC (group 1 and group 3) were considered, thus all patients received at least three courses of ddAC (a DSB-inducing regimen).

Pathology and Response Evaluation

All pre-treatment biopsies were reviewed by two pathologists. ER and PR percentages were determined by immunohistochemistry (IHC), and HER2 was assessed by IHC and CISH. For some analysis ER and PR were dichotomized as percentage lower than 50% or higher (variable names: ER_{—}50, PR_{—}50). Pre-treatment lymph node status was assessed at pathology. The response of the primary tumor to chemotherapy was evaluated by contrast-enhanced MRI^{[21]} after 3 courses of chemotherapy, and after completion of chemotherapy by pathologic evaluation of the resection specimen. The primary end point of both studies was a pCR, defined as the complete absence of residual invasive tumor cells seen at microscopy. If only non-invasive tumor (carcinoma in situ) was detected, this was considered a pCR as well. When a small number of scattered tumor cells were seen, the samples were classified as ‘near pCR’ (npCR). Because the aim of this study was to determine if HRD was correlated with a higher sensitivity to chemotherapy, tumors with a npCR were included in the group of complete remission for analytical purposes. Patients with larger amounts of residual tumor left were classified as non-complete responders (NR).

Array-CGH

Tumor DNA and reference DNA were co-hybridized using two different CyDyes to a microarray containing 3.5 k BAC/PAC derived DNA segments covering the whole genome with an average spacing of 1 MB and processed as described before^{[22]}. Classification of subtypes was performed using an aCGH BRCA1 and BRCA2 classifier^{[5] [23]}. In this Example, the same classifier used in the preceding Example (FIG. 2) was utilized and a BRCA1 probability score ≧0.63 was considered as a BRCA1-like aCGH pattern^{[19]}. Under this cut-off a tumour was called sporadic-like. The cut-off for a BRCA2-like aCGH pattern was 0.5, as described previously^{[23]}.

RT-PCR

mRNA isolation and extraction were performed using RNA Bee, according to the manufacturer's protocol (Isotex, Friendswood, Tex.). A 5 μm section halfway through the biopsy was stained for Hematoxylin and Eosin and analyzed by a pathologist for tumor cell percentage. Only samples that contained at least 60% tumor cells were included in the further analysis. RT-qPCR was performed using TaqMan Pre-designed gene expression Assay for BRCA1 (#Hs01556193). The standard curve method was used. GAPDH and B-actin were measured for normalization purposes and the average of both gene expression values was used. The cut-off between BRCA1 low and normal gene expression was 0.25. This cut-off was empirically determined.

MLPA

Hypermethylation of the BRCA1 promoter was determined using a custom Methylation specific MLPA set, according to the manufacturers' protocol (MRC-Holland; ME005-custom). When the two BRCA1 markers both showed methylation, the BRCA1 promoter was considered to be methylated. Amplification of EMSY (C11orf30) was determined using a custom MLPA set, containing seven different EMSY probes and nine reference probes (MRC Holland; X025). This EMSY MLPA set was first validated by an EMSY FISH assay (Dako). From the comparison of the EMSY FISH assay and the MLPA, it was concluded that an average of the seven probes above 1.5 corresponded to EMSY amplification, as detected by at least 6 copies of the probe at the FISH assay. DNA fragments were analyzed on a 3730 DNA Analyzer (AB, USA). Probe sequences for both MLPA kits are available on request (info@mlpa.com). For normalization and analysis the Coffalyzer program was used (MRC-Holland).

Statistical Tests

The Fisher's exact test was used to assess association between the dichotomized HRD characteristics, pathological and clinical variables. Logistic regression was performed to adjust for the following variables: age, T-stage, N-stage, ER percentage, PR percentage. All data analyses were performed using SPSS version 17.

Overview of Samples

The frequency of HRD characteristics was studied in pre-treatment biopsies, and subsequently the findings were related to neoadjuvant chemotherapy response. A total of 60 triple negative (TN) and 103 ER+ HER2− tumors were studied, which all received neoadjuvant chemotherapy with doxorubicin and cyclophosphamide (AC-regimen). Table 4 shows the clinical pathological characteristics of all tumors. The majority of the tumors were T-stage 2 or 3 and lymph node positive. Most patients were treated by 6× ddAC, although some switched to the DC regimen after 3 courses of AC. TN tumors had a higher percentage of responders (pCR+npCR) than ER+ patients. Table 5 gives the frequencies of the HRD characteristics per tumor group. BRCA1-related abnormalities (aCGH BRCA1-like profile, BRCA1 promoter methylation and low BRCA1 mRNA expression) were predominantly observed in the TN tumors (table 5). The percentage of aberrations was not different between patients treated with 6 cycli of AC versus patients treated with 3 cycli AC followed by 3 cycli of DC (data not shown). As the pattern of characteristics and also the response rates to chemotherapy are different in hormone receptor positive and negative tumors, they were analyzed separately.

TABLE 4
Patient and tumor characteristics

| | TN | | ER+ | |
|---|---|---|---|---|
| Number of patients | 60 | | 103 | |
| Median age (sd) | 42 (11.8) | | 48 (8.9) | |
| Progesterone receptor | | | | |
| Positive | | | 70 | 68% |
| Negative | 60 | 100% | 32 | 31% |
| NA | | | 1 | 1% |
| T-stage | | | | |
| T1 | 3 | 5% | 12 | 12% |
| T2 | 42 | 70% | 56 | 54% |
| T3 | 10 | 17% | 31 | 30% |
| T4 | 5 | 8% | 4 | 4% |
| N-stage | | | | |
| Node negative | 23 | 38% | 16 | 16% |
| Node positive | 37 | 62% | 87 | 84% |
| Chemotherapy | | | | |
| 6 × ddAC | 51 | 85% | 81 | 79% |
| 3 × ddAC, 3 × DC | 9 | 15% | 22 | 21% |
| Response | | | | |
| pCR | 21 | 35% | 12 | 12% |
| npCR | 10 | 17% | 12 | 12% |
| PR + NR | 27 | 45% | 77 | 75% |
| unknown | 2 | 3% | 2 | 2% |

(n)pCR = (near) pathological complete remission; PR + NR = partial and non response ddAC = dose dense doxorubixin cyclophosphamide, DC= docetaxel, capecitabine

TABLE 5
Summary of HRD characteristics

| | | ER+ | p- |
|---|---|---|---|
| | TN (n = 60) | (n = 103) | value |
| aCGH BRCA1 like | | | |
| BRCA1 like | 34 (57%) | 6 (6%) | |
| Sporadic like | 26 (43%) | 97 (94%) | <0.001 |
| BRCA1expression | | | |
| low | 13 (22%) | 2 (2%) | |
| normal/high | 23 (38%) | 58 (56%) | <0.001 |
| Not determined | 24 (40%) | 43 (42%) | |
| BRCA1 promotor methylation | | | |
| Methylated | 12 (20%) | 1 (1%) | |
| Unmethylated | 37 (62%) | 55 (53%) | <0.001 |
| Not determined | 11 (18%) | 47 (46%) | |
| EMSY Amplification | | | |
| Amplification | 2 (3%) | 11 (11%) | |
| Retention | 34 (57%) | 72 (70%) | 0.339 |
| Not determined | 24 (40%) | 20 (19%) | |

*Due to limited biopsy material, methylation, gene expression and EMSY amplification were not performed on all samples.

TN Tumors and BRCA1-Related Abnormalities

The BRCA1-like aCGH profile was predominantly seen in TN tumors (57% in TN vs 6% in ER+ tumors, p<0.001), (table 5). Other features of BRCA1 inactivation were assessed by determination of BRCA1 promoter methylation and the level of BRCA1 mRNA expression. These two characteristics were again predominantly observed in TN tumors, but were less frequent than a BRCA1-like aCGH pattern: 25% of TN tumors showed BRCA1 promoter methylation and 36% of TN tumors showed a low BRCA1 gene expression.

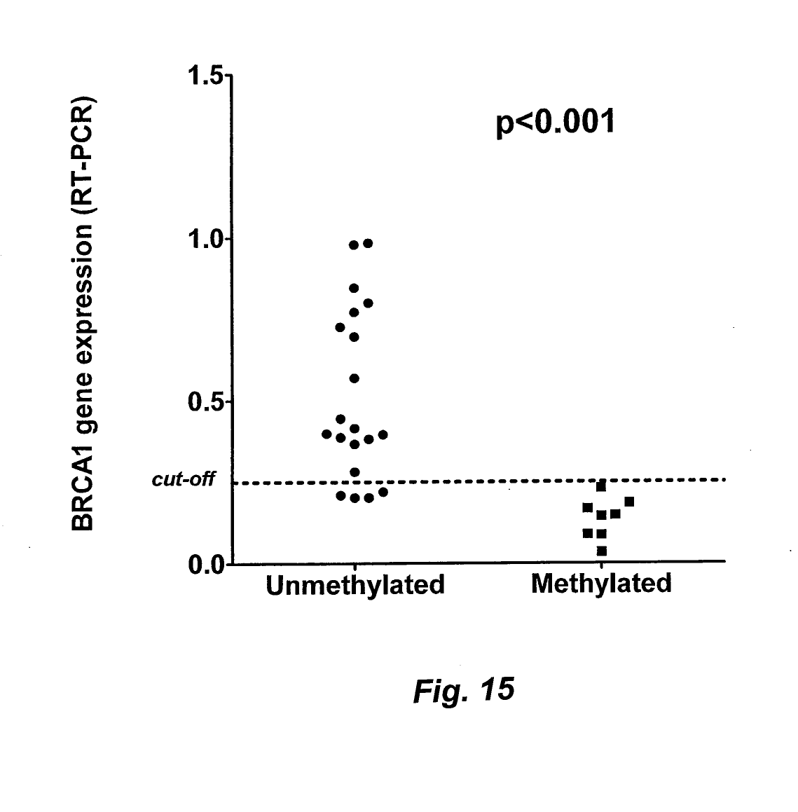

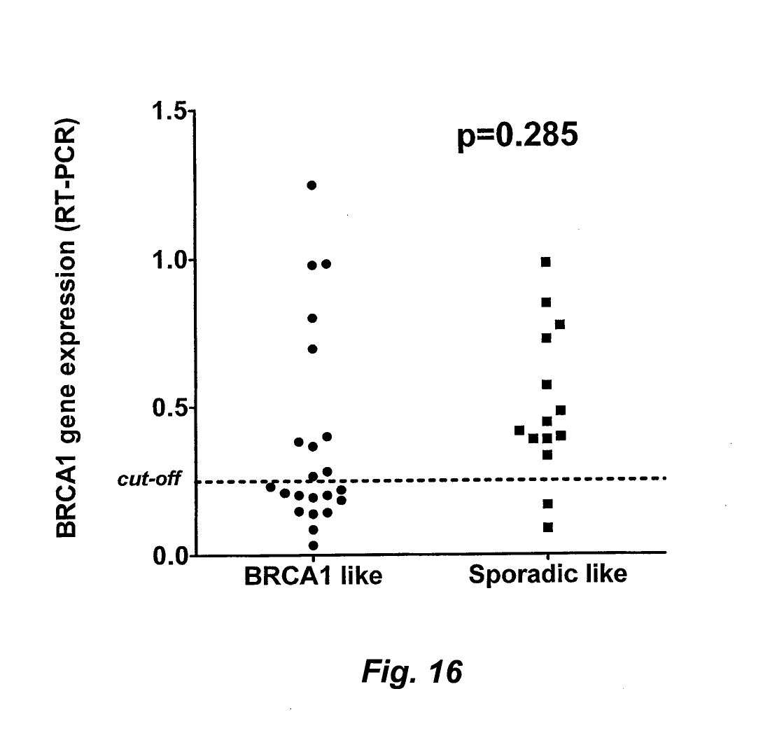

The relation between the three BRCA1-related abnormalities was subsequently determined. FIGS. 15 and 16 show the relation between mRNA expression, methylation and a BRCA1-like aCGH pattern. The cut-off between low and normal BRCA1 gene expression was empirically determined based on methylation status. It was assumed that methylated samples would have a low mRNA expression, so the cut-off was set at 0.25 (FIG. 15). All methylated samples therefore have, by definition, a low BRCA1 gene expression. The median mRNA gene expression of methylated samples was 0.156 while unmethylated samples show a value of 0.398. This difference was statistically significant (p<0.001). The relation between the BRCA1-like aCGH pattern and BRCA1 mRNA expression was also studied (FIG. 16), as low gene expression could be expected to be associated with a BRCA1-like aCGH pattern. Indeed, most BRCA1-like samples have a low expression of the BRCA1 gene, whereas sporadic-like samples have more frequently a normal mRNA expression level. Samples with a BRCA1-like aCGH profile have a median mRNA expression of 0.226, while sporadic-like samples have a median mRNA expression value of 0.426, however, this difference was not statistically significant. From the 12 tumors with BRCA1 promoter methylation, 8 had a BRCA1-like aCGH pattern and 4 a sporadic-like aCGH pattern.

Next, the association between BRCA1 inactivation and clinical and pathological variables and response to chemotherapy with DSB causing agents was studied. There was no difference in T-stage or N-stage between tumors with BRCA1-alterations and without (table 6). Patients with tumors showing BRCA1 methylation were younger than those with non-methylated tumors. Treatment response on A/C was not different between tumors with BRCA1 alterations and without these alterations: 58% vs. 48%, (p=0.47) for BRCA1-like vs. a sporadic-like aCGH profile; 55% vs. 61% (p=0.70) for methylated vs. unmethylated tumors and 54% vs. 61% (p=0.68) for low gene expression vs. normal gene expression.

TABLE 6
Clinical and pathological characteristics according to BRCA1 alterations in TN tumors.

| | BRCA1-like aCGH | | | | | | | | | | BRCA1 gene expression | | | | |
|---|---|---|---|---|---|---|---|---|---|---|---|---|---|---|---|
| | Sporadic | | BRCA1 | | | BRCA1 methylation | | | | | normal | | low | | |
| | like | | like | | P- | Unmethylated | | Methylated | | P- | mRNA | | mRNA | | P- |
| Variable | N | % | N | % | value | N | % | N | % | value | N | % | N | % | value |
| T_stage | | | | | | | | | | | | | | | |
| T½ | 20 | 77 | 25 | 74 | | 27 | 73 | 10 | 83 | | 18 | 78 | 10 | 77 | |
| T¾ | 6 | 23 | 9 | 26 | 0.76 | 10 | 27 | 2 | 17 | 0.47 | 5 | 22 | 3 | 23 | 0.93 |
| N_stage | | | | | | | | | | | | | | | |
| LN neg | 10 | 38 | 13 | 38 | | 16 | 43 | 4 | 33 | | 8 | 35 | 4 | 31 | |
| LN pos | 16 | 62 | 21 | 62 | 0.99 | 21 | 57 | 8 | 67 | 0.54 | 15 | 65 | 9 | 69 | 0.81 |
| Age | | | | | | | | | | | | | | | |
| <=40 | 10 | 38 | 19 | 56 | | 15 | 41 | 11 | 92 | | 9 | 39 | 8 | 62 | |
| >40 | 16 | 62 | 15 | 44 | 0.18 | 22 | 59 | 1 | 8 | 0.002 | 14 | 61 | 5 | 38 | 0.2 |
| Response | | | | | | | | | | | | | | | |
| PR + NR | 13 | 50 | 14 | 41 | | 14 | 38 | 5 | 42 | | 9 | 39 | 6 | 46 | |
| pCR + npCR | 12 | 46 | 19 | 56 | 0.47 | 22 | 59 | 6 | 50 | 0.7 | 14 | 61 | 7 | 54 | 0.68 |
| Unknown | 1 | 4 | 1 | 3 | | 1 | 3 | 1 | 8 | | | | | | |

In the series of patients described in this Example, the frequency of certain features associated with homologous recombination deficiency (HRD) was studied in untreated breast cancers and possible relationships with neoadjuvant treatment response were explored. This study was restricted to HER2-negative tumors, as the focus of study was the effect of DNA double strand break (DSB)-inducing agents unperturbed by the effect of targeted therapy such as Traztuzumab. In TN tumors we found mainly BRCA1-related abnormalities.

In TN tumors, no difference in response rates was observed between patients with BRCA1-like aCGH tumors and tumors with a sporadic-like aCGH pattern. In the study presented in Example 1, the BRCA1-like aCGH pattern was shown to be associated with an important survival benefit of intensive treatment with platinum-based chemotherapy for high-risk primary breast cancer^{[19]}. It is possible that any hypersensitivity to DSB inducing agents only shows at higher doses, while the lower standard dose causes increased genomic instability rather than cell death.

In a recent report by Kriege et al, it was shown that BRCA2 hereditary breast cancers were more sensitive to chemotherapy with anthracyclines or CMF than sporadic breast cancers^{[24]}. For BRCA1 hereditary breast cancer, there was no significant difference in sensitivity. The authors explain the difference in outcome between BRCA1- and BRCA2-mutated tumors by different tumor characteristics, including higher grade, triple negativity and a higher incidence of p53 mutations. The finding presented in this Example, that aberrations in BRCA1 are characteristic for TN tumors, is in line with this. BRCA1-mutated tumors are usually basal like or triple negative.

In conclusion, in TN tumors, BRCA-ness occurred in about half of all cases, but did not predict a better treatment response to standard dose chemotherapy with AC. It is certainly possible that conventional doses of cisplatin or carboplatin would be highly effective in this subgroup, as suggested in the literature^{[20]}.

### REFERENCES

1. Rastogi P, Anderson S J, Bear H D et al. Preoperative chemotherapy: updates of National Surgical Adjuvant Breast and Bowel Project Protocols B-18 and B-27. J Clin Oncol 2008; 26: 778-785.
2. van der Hage J A, van de Velde C J, Julien J P et al. Preoperative chemotherapy in primary operable breast cancer: results from the European Organization for Research and Treatment of Cancer trial 10902. J Clin Oncol 2001; 19: 4224-4237.
3. Gianni L, Baselga J, Eiermann W et al. Feasibility and tolerability of sequential doxorubicin/paclitaxel followed by cyclophosphamide, methotrexate, and fluorouracil and its effects on tumor response as preoperative therapy. Clin Cancer Res 2005; 11: 8715-8721.
4. Sachelarie I, Grossbard M L, Chadha M et al. Primary systemic therapy of breast cancer. Oncologist 2006; 11: 574-589.
5. Joosse S A, van Beers E H, Tielen I H et al. Prediction of BRCA1-association in hereditary non-BRCA1/2 breast carcinomas with array-CGH. Breast Cancer Res Treat 2009; 116: 479-489.
6. Turner N, Tutt A, Ashworth A. Hallmarks of ‘BRCAness’ in sporadic cancers. Nat Rev Cancer 2004; 4: 814-819.
7. Hughes-Davies L, Huntsman D, Ruas M et al. EMSY links the BRCA2 pathway to sporadic breast and ovarian cancer. Cell 2003; 115: 523-535.
8. Kennedy R D, Quinn J E, Mullan P B et al. The role of BRCA1 in the cellular response to chemotherapy. J Natl Cancer Inst 2004; 96: 1659-1668.
9. Rottenberg S, Nygren A O, Pajic M et al. Selective induction of chemotherapy resistance of mammary tumors in a conditional mouse model for hereditary breast cancer. Proc Natl Acad Sci USA 2007; 104: 12117-12122.
10. Rottenberg S, Jaspers J E, Kersbergen A et al. High sensitivity of BRCA1-deficient mammary tumors to the PARP inhibitor AZD2281 alone and in combination with platinum drugs. Proc Natl Acad Sci USA 2008; 105: 17079-17084.
11. Ratnam K, Low J A. Current development of clinical inhibitors of poly(ADP-ribose) polymerase in oncology. Clin Cancer Res 2007; 13: 1383-1388.
12. O'Shaughnessy J, Osborne C, Pippen J et al. Efficacy of BSI-201, a poly (ADP-ribose) polymerase-1 (PARP1) inhibitor, in combination with gemcitabine/carboplatin (G/C) in patients with metastatic triple-negative breast cancer (TNBC): Results of a randomized phase II trial. J Clin Oncol (Meeting Abstracts) 2009; 27: 3.
13. Fong P C, Boss D S, Yap T A et al. Inhibition of poly(ADP-ribose) polymerase in tumors from BRCA mutation carriers. N Engl J Med 2009; 361: 123-134.
14. Waddell N, Arnold J, Cocciardi S et al. Subtypes of familial breast tumours revealed by expression and copy number profiling. Breast Cancer Res Treat 2009.
15. Tirkkonen M, Johannsson O, Agnarsson B A et al. Distinct somatic genetic changes associated with tumor progression in carriers of BRCA1 and BRCA2 germ-line mutations. Cancer Res 1997; 57: 1222-1227.
16. Stefansson O A, Jonasson J G, Johannsson O T et al. Genomic profiling of breast tumours in relation to BRCA abnormalities and phenotypes. Breast Cancer Res 2009; 11: R47.
17. Jonsson G, Naylor T L, Vallon-Christersson J et al. Distinct genomic profiles in hereditary breast tumors identified by array-based comparative genomic hybridization. Cancer Res 2005; 65: 7612-7621.
18. Wessels L F, van Welsem T, Hart A A et al. Molecular classification of breast carcinomas by comparative genomic hybridization: a specific somatic genetic profile for BRCA1 tumors. Cancer Res 2002; 62: 7110-7117.
19. Vollebergh M A, Lips E H, Nederlof P M et al. An aCGH classifier derived from BRCA1-mutated breast cancer and benefit of high-dose, platinum-based, chemotherapy in breast cancer patients. Submitted for publication 2010.
20. Silver D P, Richardson A L, Eklund A C et al. Efficacy of Neoadjuvant Cisplatin in Triple-Negative Breast Cancer. J Clin Oncol 2010.
21. Loo C E, Teertstra H J, Rodenhuis S et al. Dynamic contrast-enhanced MRI for prediction of breast cancer response to neoadjuvant chemotherapy: initial results. AJR Am J Roentgenol 2008; 191: 1331-1338.
22. Joosse S A, van Beers E H, Nederlof P M. Automated array-CGH optimized for archival formalin-fixed, paraffin-embedded tumor material. BMC Cancer 2007; 7: 43.
23. Joosse S A, Brandwijk K I, Devilee P et al. Prediction of BRCA2-association in hereditary breast carcinomas using array-CGH. Breast Cancer Res Treat 2010.
24. Kriege M, Seynaeve C, Meijers-Heijboer H et al. Sensitivity to first-line chemotherapy for metastatic breast cancer in BRCA1 and BRCA2 mutation carriers. J Clin Oncol 2009; 27: 3764-3771.
25. Raouf A, Brown L, Vrcelj N et al. Genomic instability of human mammary epithelial cells overexpressing a truncated form of EMSY. J Natl Cancer Inst 2005; 97: 1302-1306.
26. Trudeau M E, Pritchard K I, Chapman J A et al. Prognostic factors affecting the natural history of node-negative breast cancer. Breast Cancer Res Treat 2005; 89: 35-45.
27. Fisher E R, Wang J, Bryant J et al. Pathobiology of preoperative chemotherapy: findings from the National Surgical Adjuvant Breast and Bowel (NSABP) protocol B-18. Cancer 2002; 95: 681-695.

Finally, it should be noted that there are alternative ways of implementing the embodiments disclosed herein. Accordingly, the present embodiments are to be considered as illustrative and not restrictive. Furthermore, the claims are not to be limited to the details given herein, and are entitled their full scope and equivalents thereof.

1. A method for optimizing the therapeutic efficacy of anti-cancer therapy in a patient, comprising:
obtaining a cell sample from the patient;
detecting the copy numbers of genomic DNA in the patient's cell sample in at least 3 genomic loci selected from 1p35-21, 3q22-27, 5p13, 5q21-34, 6p25-22, 7p21-15, 7q31-36, 8q22-24, 10p15-14, 10p12, 12p13,12q21-23, 13q31-33, 14q22-24, 15q14-21 and 21q11-22; and
comparing the copy numbers in the patient's cell sample to corresponding copy numbers in a non-cancerous cell sample;
wherein a variation in the copy numbers in the patient's cell sample classifies the cell sample as from a BRCA1-associated tumor and indicates that the patient will benefit from anti-cancer therapy.

2.-3. (canceled)

4. A method according to claim 1, wherein the array comprises at least three of the BAC probes of FIG. 2.

5. The method of claim 4, wherein the array comprises at least 10 of the BAC probes of FIG. 2.

6. The method of claim 4, wherein the BRCA1 array comprises at least 50 of the BAC probes of FIG. 2.

7. The method of claim 4, wherein the BRCA1 array comprises at least 100 of the BAC probes of FIG. 2.

8. The method of claim 4, wherein the BRCA1 array comprises the BAC probes of FIG. 2.

9. A method according to claim 1, wherein the cancer therapy is intensive alkylator-based chemotherapy.

10. A BRCA1 array comprising at least three of the BAC probes of FIG. 2.

11. A BRCA1 array according to claim 10, wherein the array comprises the BAC probes of FIG. 2.

12. A BRCA1 array according to claim 10, said array capable of detecting the copy numbers of genomic DNA in at least three of the genomic loci selected from 1p35-21, 3q22-27, 5p13, 5q21-34, 6p25-22, 7p21-15, 7q31-36, 8q22-24, 10p15-14, 10p12, 12p13, 12q21-23, 13q31-33, 14q22-24, 15q14-21 and 21q11-22.

13. A BRCA1 array according to claim 10, said array capable of detecting the copy numbers of genomic DNA in at least three of the genomic loci selected from 1p35.1-21.3, 3q22.2-27.2, 5p13.2, 5q21.3-34, 6p25.2-22.1, 7p21.3-15.3, 7q31.33-36.3, 8822.1-24.3, 10p15.3-14, 10p12.1, 12p13.33-13.2, 12q21.2-23.3, 13q31.2-33.3, 14q22.1-24.1, 15q14-21.1 and 21q11.2-22.3.

14. A method of assessing anti-cancer therapies for breast cancer, comprising:
obtaining a cell sample from the patient;
detecting the copy numbers of genomic DNA in the patient's cell sample in at least 3 genomic loci selected from 1p35-21, 3q22-27, 5p13, 5q21-34, 6p25-22, 7p21-15, 7q31-36, 8q22-24, 10p15-14, 10p12, 12p13, 12q21-23, 13q31-33, 14q22-24, 15q14-21 and 21q11-22; and
comparing the copy numbers in the patient's cell sample to corresponding copy numbers in a non-cancerous cell sample;
wherein a variation in the copy numbers in the patient's cell sample classifies the cell sample as from a BRCA1-associated tumor and indicates that the patient will benefit from the anti-cancer therapies.

15.-16. (canceled)
